(19) United States
(12) Patent Application Publication
(10) Pub. No.: US 2024/0167101 A1
(43) Pub. Date: May 23, 2024

(54) GENE EXPRESSION ANALYSIS FOR CANCER IMMUNOTHERAPY

(71) Applicant: THE CLEVELAND CLINIC FOUNDATION, Cleveland, OH (US)

(72) Inventor: Brian Gastman, Beachwood, OH (US)

(21) Appl. No.: 18/514,400

(22) Filed: Nov. 20, 2023

Related U.S. Application Data
(60) Provisional application No. 63/426,819, filed on Nov. 21, 2022.

(51) Int. Cl. C12Q 1/6886; C12Q 1/6874

(52) U.S. Cl. CPC C12Q 1/6886; C12Q 1/6874; C12Q 2600/106; C12Q 2600/158

(57) ABSTRACT

A method of treating cancer in a subject in need thereof is described. The method includes determining the level of expression of a signature gene in a lymphocyte sample from the subject; and selecting the type of cancer treatment for the subject based on the differential level of expression of one or more signature genes.

### CROSS-REFERENCE TO RELATED APPLICATIONS

This application claims priority to U.S. Provisional Patent Application Ser. No. 63/426,819, filed Nov. 21, 2022, which is incorporated herein by reference.

### BACKGROUND

Approximately 50% of melanoma patients respond to immunotherapy. However, even responders can relapse and then become resistant to their treatment regimen. In addition, there is a large group of patients who display resistance at the outset of treatment.

Unfortunately, physicians and health care providers must wait until there are either physical symptoms or diagnostic imaging results in order to assess treatment outcome. Current techniques to assess the efficacy of immunotherapy generally involve highly invasive tissue sampling, and not all patients are able to safely undergo such biopsies. This gene expression analysis of peripheral blood lymphocytes will allow for a non-invasive, earlier diagnosis, and will overcome the limitations of current methods. Additionally, an ability to assess the efficacy of immunotherapy at an earlier time point will allow a change of intervention, if necessary, which will likely allow for improved survival and outcomes.

The development of immune checkpoint inhibitor (ICI) therapies, including anti-programmed cell death 1 (PD-1)/programmed death-ligand 1 (PD-L1) and anti-cytotoxic T lymphocyte antigen 4 (CTLA4), represents a major advance in the treatment of patients with melanoma. However, not all patients respond to these therapies (Garon et al., J. Clin. Oncol. 37:2518-2527 (2019)), indicating that additional strategies are needed to improve responses. Among the immune repertoire, CD8 T cells play critical roles in ICI therapies and thus are an important cell type to focus on to improve immunotherapy. Wei et al., Cell. 170:1120-1133.e17 (2017).

We and others showed that tumor-infiltrating lymphocytes (TILs), including CD8 T cells, express high levels of immune checkpoint receptors that include PD-1, which can suppress cytotoxic T cell effector function. Maybruck et al., J. Immunother. Cancer. 5:65 (2017); Pfannenstiel et al., Cancer Immunol. Res. 7:510-525 (2019). These TILs often display so-called exhausted behaviors characterized by antigen unresponsiveness and reduced cytotoxic effector function. Sharma et al., Cell. 168:707-723 (2017). While therapeutic blockade of PD-1/PD-L1 and/or CTLA4 can revive effector T cell function, this is not always complete and may explain the lack of anti-tumor therapeutic efficacy (Larkin et al., N. Engl. J. Med. 381:1535-1546 (2019).

Importantly, CD8 T cell tumor infiltration remains one of the most correlated factors for anti-PD-1/PD-L1 immunotherapy response across cancer types, as well as tumor mutational burden and high PD-1/PD-L1 expression levels. Samstein et al., Nat. Genet. 51:202-206 (2019). Today, no predictive model of ICI response exists that is robust enough to implement in the treatment algorithm for melanoma patients. Liu et al., Nat. Med. 25:1916-1927 (2019). Other studies have focused on gene expression and transcriptomic data of T cells to predict patient response to anti-PD-1 therapy. Auslander et al., Nat. Med. 24:1545-1549 (2018); these strategies required invasive biopsies and resulted in moderate prognostic value.

### SUMMARY OF THE INVENTION

Data from the National Cancer Institute (NCI) surgery branch indicate that there are overlapping CD8 subpopulations both in the TIL and in the periphery, which was recently corroborated at the transcriptomic level. Wu et al., Nature. 579:274-278 (2020). Comparing the inventors' studies of tumor-associated dysfunctional CD8 T cells with suppressor cell function and those from the NCI surgery branch (both studies identified PD-1 expression as critical) led them to hypothesize that this population exists in both the tumor and peripheral blood (Gros et al., J. Clin. Invest. 129:4992-5004 (2019).

Using single RNA from patient's peripheral blood lymphocytes and tumor infiltrating lymphocyte the inventors have developed Gene Expression Profile(s) (GEP) that can be used to distinguish responder from non-responder populations. This will help care providers better monitor, manage, and prognose patients with cancer such as melanoma.

Using single cell RNA sequencing analysis from patient's peripheral blood lymphocytes and tumor infiltrating lymphocyte we have developed Gene Expression Profile(s) (GEP) that consist of 28 signature genes that can be used to distinguish responder from non-responder populations. This will help care providers better monitor, manage, and prognose patients with melanoma. The identification of GEP does not depend on one unique platform, but could be determined using any bioinformatic analyses. Importantly, leveraging on a new PrimeFlow assay, an innovative flow cytometry-based technique incorporating RNA hybridization, the inventors have verified this GEP is capable of stratifying non-responders from responders. In their pilot assay, they have proven the feasibility of translating this GEP into clinical use for predicting response to cancer immunotherapy.

### BRIEF DESCRIPTION OF THE FIGURES

The present invention may be more readily understood by reference to the following figures, wherein:

FIG. 1 provides a schematic overview of the study. Single-cell transcriptomic analyses of CD8 T cells in PBLs (CD8-mPBL) and TILs (CD8-mTIL) from eight melanoma patients revealed three distinct shared cell subsets and unique genetic programming in both CD8 PBLs and TILs that possessed high-OXPHOS dysfunctional and inactive dormant polarization. The figure shows the development of a novel immunotherapy response predictive model (NiCir) using a PD-1 co-expression gene profile, cancer-induced molecular programming, and transcriptome results.

FIGS. 2A-2E provide graphs showing the scRNaseq profiles of 173,061 CD8 T cells from eight melanoma patients' PBLs (CD8-mPBL) and TILs (CD8-mTIL). (A) Correlation of CD8 T cell proportions in PBLs and TILs from eight melanoma patients (ID #1-8). (B) t-SNE clustering of scRNaseq data of CD8-mPBL and CD8-mTIL from melanoma patients. (C) Distribution of clusters in CD8-mPBL or CD8-mTIL. (D) Proportion of CD8-mPBL and CD8-mTIL in each cluster. (E) Pathways enriched in CD8-mPBL or CD8-mTIL; analyses were conducted with whole-transcriptome using QIAGEN IPA. The log P value, z score, and dot size represent significance and activity of pathway enrichment and the number of genes found in that pathway, respectively.

FIGS. 3A-3F provide graphs and images showing the characteristics of the three clusters in 173,061 peripheral and tumor-infiltrating CD8 T cells from eight melanoma patients. (A) Among a total of 20 major clusters, three (clusters 2, 6, and 15) contained comparable proportions of cells from CD8-mPBLs and CD8-mTILs. (B) Similarities of the three shared clusters to each of the other clusters (the top 50% nearest mathematical distances between clusters are shown as ribbons). (C) Heatmap of signature genes for each cluster of the overall datasets showing 472 genes that were significantly enriched in at least one cluster. (D) Violin plots showing expression levels of representative signature genes in shared clusters (c2, c6, and c15). (E) Pathways enriched in clusters 2, 6, and 15; analyses were conducted with whole-transcriptome using QIAGEN IPA. The log P value, z score, and dot size represent significant activity of pathway enrichment and the number of genes found in that pathway, respectively. (F) Enrichment of exhausted/dysfunctional markers in the CD8 PBL/TIL shared cluster 15. c, cluster; NER, nucleotide excision repair.

FIGS. 4A-4E provide graphs and images showing the trajectory analyses reveal distinct cell fate of shared blood/tumor-infiltrating CD8 T cells. Trajectories were constructed using equal number (1,000) of peripheral and tumor-infiltrating CD8 T cells from each patient. (A) Trajectory position of CD8-mPBLs and CD8-mTILs along pseudotime branches. (B and C) Three common clusters in CD8-mPBLs and CD8-mTILs are located at the extreme end of each branch of the tripod-shaped trajectory plot of all combined samples (B) or CD8-mPBLs or CD8-mTILs (C) on the whole-transcriptome trajectory consisting of 16,000 cells. (D) Heatmap showing genes that had significant variations along the two branches of the trajectory, which were identified by the tool Monocle; each row represents one gene. (E) Pathway activation distribution along the three trajectory branches.

FIGS. 5A-5C provide a schematic representation of cancer-induced programs in peripheral and tumor-infiltrating CD8 T cells from eight melanoma patients. (A) Schematic summary of cluster distributions along the trajectory branches and the three proposed cell programs. (B) Unique GEP of transcription regulators, membrane receptors, and cytokines along programs 1 (cluster 2→cluster 15), 2 (cluster 2→cluster 6), and 3 (cluster 15→cluster 6) in CD8 T cells in melanoma patients. (C) GEP of multiple checkpoint and TOX along the three proposed cell programs. c, cluster.

FIGS. 6A-6H provide graphs and schematics showing the cell metabolism landscape of CD8 T cells in melanoma. (A) Distribution of representative signature genes in glycolysis, OXPHOS, glucose, and lipid transportation by trajectory analyses. (B-D) t-SNE plots showed activation of glycolysis and OXPHOS pathways in peripheral or tumor CD8 T cells (B) or along trajectories of PBLs and TILs (C and D). (E) Transition of cell metabolism along the pseudotime development, with arrows indicating increasing activity. (F) z score of T cell exhaustion signaling versus OXPHOS signaling of the T cell clusters. (G) CD8+ PD-1+CD38+CD39+ T cell cluster with high OXPHOS level (CD8+ TOXPHOS) on the trajectories in CD8-mPBL and CD8-mTIL. (H) z score of CD38 and CD39 expression versus OXPHOS signaling of the T cell clusters.

FIGS. 7A-7F provide graphs showing the high bioenergy of CD8 T cells in PBLs of refractory melanoma. (A) Glucose OCR of CD8+ PD-1+ TILs was higher in melanoma compared with CD8 T cells of healthy PBLs by seahorse assay (in triplicate). (B) Glucose dependency rate of melanoma TILs was significantly elevated (in triplicate). (C) High ATP level of CD8 T cells in nonresponders for TILs and PBLs (PBLs: naive n=5, responder n=5, nonresponder n=5; TILs: naive n=4, nonresponder n=4). (D) Schematic representation of flow cytometry of TMRM showing low TMRM (TMRMlo) and high TMRM (TMRMhi) for naive (left) and nonresponder patients (right) in TILs. (E) Mitochondria activity measured by normalized TMRMhi MFI of PD-1+, PD-1+CD39+, and PD-1+CD38+CD39+ T cell subpopulations in PBL CD8 T cells from naive (n=4), responder (n=5), or nonresponder (n=7) patients. Nonresponders showed significant higher normalized MFI in peripheral blood. (F) Significant elevation of mitochondria activity by normalized TMRMhi MFI in PD-1+CD39+ and PD-1+CD38+CD39+ T cells of nonresponder (n=6) patients in TIL CD8 T cells as compared with naive cells (n=5). TMRMhi MFIs in E and F were normalized to intensity of MitoTracker as an indicator of mitochondrial mass. *, P<0.05; **, P<0.01; ***, P<0.001; ****, P<0.0001 unpaired t test (two tailed). Error bars represent SEM.

FIGS. 8A-8D provide graphs and images of the Immunotherapy response prediction model NiCir built on PD-1 coexpression genes. (A) Heatmap of PD-1 and the top 20 coexpressed genes of CD8-mPBL-or CD8-mTIL-dominated clusters and shared clusters. (B) Gene ontology enrichment of the top 1,000 genes correlated mostly with PD-1 expression in the three shared clusters. (C) Performances of NiCir's prediction in the training dataset (GSE120575) of melanoma patients from a previously published study and three additional validation datasets: one scRNaseq dataset of nonmelanoma skin cancer from a previous study (dataset 1, GSE123813) and melanoma PBL samples collected at the Cleveland Clinic core (dataset 2, GSE138720 and GSE153098; and dataset 3, GSE152590 and GSE171256). The P values of NRSs between true responders and true non-responders were calculated by t test (P<0.05 as significant). (D) Performance of NiCir on the validation datasets indicated by receiver-operating characteristic (ROC) curve and AUC value for 51 samples.

### DETAILED DESCRIPTION OF THE INVENTION

The present invention provides a method of treating cancer in a subject in need thereof. The method includes determining the level of expression of a signature gene in a lymphocyte sample from the subject; and selecting the type of cancer treatment for the subject based on the level of expression of one or more signature genes.

### Definitions

“Diagnosis” as used herein generally includes determination as to whether a subject is likely affected by a given disease, disorder or dysfunction. The skilled artisan often makes a diagnosis on the basis of various symptoms and/or one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in symptoms.

As used herein, the term “gene” refers to a nucleic acid fragment that expresses a specific protein, including regulatory sequences preceding (5′ non-coding sequences) and following (3′ non-coding sequences) the coding sequence.

The phrase “differentially expressed” refers to differences in the quantity and/or the frequency of a biomarker present in a sample taken from patients having, for example, melanoma as compared to a control subject or subject without cancer. For example, a biomarker can be a polynucleotide which is present at an elevated level or at a decreased level in samples of patients with cancer compared to samples of control subjects. Alternatively, a biomarker can be a polynucleotide which is detected at a higher frequency or at a lower frequency in samples of patients with cancer (e.g., melanoma) compared to samples of control subjects. A biomarker can be differentially present in terms of quantity, frequency or both.

A polynucleotide is differentially expressed between two samples if the amount of the polynucleotide in one sample is statistically significantly different from the amount of the polynucleotide in the other sample. For example, a polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment or diagnosis of humans is of particular interest.

As used herein, the term “about” refers to +/−10% deviation from the basic value.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “a biomarker” includes reference to one or more biomarkers, and so forth.

### Guiding Treatment by Determining the Level of Expression of a Signature Gene in a Lymphocyte Sample

In one aspect, the present invention provides a method of treating cancer in a subject in need thereof. The method can be used for a subject who has just been diagnosed with cancer, or a subject who is already undergoing treatment for cancer. The method includes determining the level of expression of a signature gene in a lymphocyte sample from the subject and selecting the type of cancer treatment for the subject based on the differential level of expression of one or more signature genes. The differential level of expression can represent a higher or a lower level of expression as compared with the control level of expression of a given signature gene.

A signature gene is a gene that can be used to distinguish cancer immunotherapy responders from non-responders. In some embodiments, the signature genes are selected from the group consisting of TUBB, TUBA1B, HIST1H4C, HMGB2, H2AFZ, FABP5, HMGN2, HMGB1, COTL1, TPI1, CALM3, ACTB, PSMB9, CALM2, CLIC1, CD74, CST7, LSP1, SRGN, HLA-C, NKG7, LAG3, PDCD1, HLA-A, STMN1, GAPDH, PD1, and RPL13A. In some embodiments, the expression level of a plurality of signature genes is evaluated. In some embodiments, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 signature gene expression levels are evaluated.

The terms “cancer,” “tumor” and “neoplasia” are used interchangeably herein and refer to a cell or population of cells whose growth, proliferation or survival is greater than growth, proliferation or survival of a normal counterpart cell, e.g., a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. In some embodiments, the cancer is selected from the group of cancer types consisting of sarcoma, carcinoma, and lymphoma. Cancer can also be characterized based on the organ in which it is growing. Examples of cancer characterized in this fashion include bladder cancer, prostate cancer, liver cancer, breast cancer, colon cancer, skin cancer (e.g., melanoma), and leukemia.

In some embodiments, the cancer is melanoma. Melanoma is a is a type of skin cancer that develops from the pigment-producing cells known as melanocytes. Melanomas typically occur in the skin, but may rarely occur in the mouth, intestines, or eye. The primary cause of melanoma is ultraviolet light (UV) exposure in those with low levels of the skin pigment melanin.

In some embodiments, the subject has been diagnosed with melanoma. Early signs of melanoma are changes to the shape or color of existing moles or, in the case of nodular melanoma, the appearance of a new lump anywhere on the skin. Lentigo maligna is an example of an early form of melanoma. At later stages, the mole may itch, ulcerate, or bleed. Melanoma are typically diagnosed by visual inspection, dermatoscopic exam, or examination using a confocal microscope. A biopsy is often required to assist in making or confirming the examination.

### Lymphocyte Samples

The method includes determining the level of expression of a signature gene in a lymphocyte sample from a subject. As used herein, a “lymphocyte sample” refers to a sample of tissue, cells, or fluid isolated from a subject, including but not limited to, for example, urine, blood, spinal fluid, lymph fluid, saliva, milk, lymphocytes, organs, biopsies, that are or include lymphocytes. In some embodiments, the lymphocyte sample comprises a blood sample.

The biological sample may be fresh or stored. For example, lymphocyte samples may be or have been stored or banked under suitable tissue storage conditions. The lymphocyte sample may have been expressly obtained for the assays of this invention or a lymphocyte sample obtained for another purpose which can be subsampled for the assays of this invention. Preferably, lymphocyte samples are either chilled or frozen shortly after collection if they are being stored to prevent deterioration of the sample.

In some embodiments, the method further comprising the step of obtaining a lymphocyte sample from the subject. The lymphocyte sample obtained from the subject to be diagnosed is typically a blood sample, but can be any sample from bodily fluids or tissue that include lymphocytes. A “control” sample, as used herein, refers to a biological sample, such as a bodily fluid, tissue, or lymphocytes from a subject that is not diseased. That is, a control sample is obtained from a normal or healthy subject (e.g., an individual who does not have melanoma).

In certain embodiments, the biological sample is a skin sample, including the entire tumor or a portion, piece, part, segment, or fraction of the melanoma. Solid tissue samples can be obtained by surgical techniques according to methods well known in the art. A melanoma biopsy may be obtained by methods including, but not limited to an incisional biopsy, a punch biopsy, an excision biopsy, a shave biopsy, a curette biopsy, a fine needle aspirate, or a saucerization biopsy.

As described herein, a lymphocyte sample is a biological sample that includes lymphocytes. A lymphocyte is an immune cell, and including natural killer cells, T cells, and B cells (for humoral, antibody-driven adaptive immunity). In some embodiments, the lymphocytes are peripheral blood lymphocytes, which are mature lymphocytes that circulate in the blood. In further embodiments, the lymphocytes are CD8+ cells, which are cytotoxic immune cells that induce apoptosis in target cells (primarily virus-infected cells). In yet further embodiments, the lymphocytes comprise CD8+ TOXPHOS cells. TOXPHOS cells are CD8+ T cells obtained from melanoma patients with high levels of oxidative phosphorylation.

The presence and/or the level of the signature gene(s) can be determined by any now known or hereafter developed assay or method of detecting and/or determining gene expression level, for example, quantitative RT-PCR, Northern blot, real-time PCR, PCR, allele-specific PCR, pyrosequencing, SNP Chip technology, or restriction fragment length polymorphism (RFLP). The signature gene can be detected or measured by an analytic device such as a kit or a conventional laboratory apparatus, which can be either portable or stationary.

Many of the methods for determining a nucleotide sequence involve PCR. As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences. Accordingly, in some embodiments, the detecting the presence and/or level of the signature gene comprises extending a primer that hybridizes to a sequence adjacent to the polymorphic nucleotide. In some embodiments, the determining the presence and/or level of signature gene comprises hybridizing a probe to a region that includes the polymorphic nucleotide.

In some embodiments, the level of gene expression is determined using flow cytometry incorporating RNA hybridization. In further embodiments, the levels of the signature gene may be compared to the level of corresponding internal standards in the sample or samples when carrying out the analysis to quantify the amount of the gene being detected.

Prior to analysis of the level of the signature gene, it may be preferable to purify the sample. DNA extraction methods are well known to those of skill in the art. For example, the Omni™ tissue DNA purification kit contains silica-based spin-capture columns and nontoxic reagents that are designed specifically for genomic DNA extraction from tissues and cultured cells. After sample lysis the DNA is purified through spin-column capture in less than 20 minutes.

The method includes comparing the level of one or more signature genes to the corresponding control values for those genes in healthy subjects. The differential level of expression can represent a higher or a lower level of expression as compared with the control level of expression of a given signature gene. For some signature genes, only a higher level of expression is significant, whereas with other signature genes only a lower level of expression is significant with regard to cancer treatment.

Once the presence and/or levels of the variant form of the signature gene(s) have been determined, they can be displayed in a variety of ways. For example, the levels can be displayed graphically on a display as numeric values or proportional bars (i.e., a bar graph) or any other display method known to those skilled in the art. The graphic display can provide a visual representation of the amount of the signature gene being evaluated.

### Cancer Treatment

The method of the invention includes the step of selecting the type of cancer treatment for the subject based on the differential level of expression of one or more signature genes. The type of cancer treatment should be selected to provide a better likelihood of successful treatment in view of the level of expression of signature genes that has been determined for the subject. The type of cancer treatment can be selected for a subject who has not yet begun cancer treatment, or for a subject who is already undergoing cancer treatment. In some embodiments, the subject has been receiving a first type of cancer treatment, the expression of one or more signature genes is increased, and selecting the type of cancer treatment comprises selecting a different, second type of cancer treatment. In further embodiments, the first type of cancer treatment comprises immunotherapy, and the second type of cancer treatment comprises a method of cancer treatment other than immunotherapy.

Methods of cancer treatment include surgery, radiation therapy, chemotherapy, immunotherapy, radioimmunotherapy, immunomodulators, and the use of vaccines. For example, melanoma can be treated using surgery to remove the tumor, adjuvant treatment (e.g., high-dose interferon treatment), chemotherapy, targeted therapy using BRAF, C-Kit and/or NRAS inhibitors (Berger et al., G Ital Dermatol Venereol, 153(3):349-360 (2018)), immunotherapy, or radiation.

In some embodiments, the differential level of expression of one or more signature genes can be used to determine if the subject is a responder or non-responder with regard to cancer immunotherapy. Accordingly, in some embodiments, the cancer treatment comprises cancer immunotherapy. In further embodiments, the cancer treatment comprises immune checkpoint inhibitor therapy.

Cancer immunotherapy is the modulation (e.g., stimulation) of the immune system to treat cancer, improving on the immune system's natural ability to fight the disease. Examples of cancer immunotherapy include cellular immunotherapy (e.g., dendritic cell therapy, CAR-T cell therapy, and T cell receptor T cell therapy), antibody therapy (e.g., administration of nivolumab, pembrolizumab, atezolizumab, durvalab, ipilumumab, or avelumab), and cytokine therapy (e.g., administration of an interferon or interleukin). In some embodiments, the cancer immunotherapy comprises administration of anti-PD1, anti-PDL1, or anti-CTL4 antibodies.

Immune checkpoint inhibitor therapy blocks immune checkpoints affecting immune system function. Tumors can use these checkpoints to protect themselves from immune system attacks. Blockade of negative feedback signaling to immune cells thus results in an enhanced immune response against tumors. Examples of immune checkpoint inhibitor therapy includes CTLA-4 blockage, administration of PD-1 inhibitors, administration of PD-L1 inhibitors, intrinsic checkpoint blockade (CISH), and administration of oncolytic viruses.

### Formulation and Administration

The present invention provides a method for treating cancer that may include administering one or more anti-cancer compounds in a pharmaceutical composition. Examples of pharmaceutical compositions include those for oral, intravenous, intramuscular, subcutaneous, or intraperitoneal administration, or any other route known to those skilled in the art, and generally involves providing an anti-cancer compound formulated together with a pharmaceutically acceptable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

For example, the maximum tolerated dose (MTD) for anti-cancer compounds can be determined in tumor-free athymic nude mice. Agents are prepared as suspensions in sterile water containing 0.5% methylcellulose (w/v) and 0.1% Tween 80 (v/v) and administered to mice (7 animals/group) by oral gavage at doses of 0, 25, 50, 100 and 200 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

In order that the subject matter disclosed herein may be more efficiently understood, an example is provided below. It should be understood that this example is for illustrative purposes only and is not to be construed as limiting the claimed subject matter in any manner.

### EXAMPLE

### CDKN1A/p21WAF1, RB1, FLG, and HRNR Mutation Patterns Provide Insights Into Urinary Tract Environmental Exposure Carcinogenesis and Potential Treatment Strategies

The inventors hypothesized that identification of overlapping CD8 subpopulations would elucidate new genetic mechanisms that could be used to not only monitor treatment outcome but also identify future therapeutic targets. Indeed, their single-cell transcriptome comparisons between purified CD8 T cells from TILs and peripheral blood lymphocytes (PBLs) from melanoma patients identified several CD8 subpopulations and underlying genetic programs. Specifically, the inventors identified three overlapping populations in TILs and PBLs. One of the populations displayed unexpectedly high levels of cytotoxic and exhausted markers (e.g., PD-1), as well as increased levels of metabolic activities, specifically oxidative phosphorylation (OXPHOS). These high-OXPHOS CD8 T cells also have elevated levels of CD38/CD39 ectonucleotidases (referred to as CD8+ TOXPHOS cells).

To further validate their findings, the inventors isolated PBL and TIL CD8+ TOXPHOS cells from melanoma patients and confirmed that they are more pronounced in ICI-resistant patients with elevated bioenergetics, including increased glucose metabolism, ATP production, and mitochondria activity. They then generated a predictive ICI therapy response model using these CD8+ TOXPHOS cells that can be assessed by either TILs or PBLs. Ultimately, they generated a gene expression profile (GEP), which was validated using both published clinically annotated transcriptomic data of CD8 TILs and a new cohort of our patients, including interrogating their CD8 PBLs. Thus, through comprehensive and careful gene expression analysis of individual CD8 T cells, it was established that an ICI response GEP that can be employed via a blood-based approach. This work establishes an ICI-predictive platform, and the CD8 subpopulation that forms the basis of this assay illustrates new targetable pathways to potentially enhance immunotherapy and improve outcomes of melanoma patients.

### Results

High Heterogeneity of CD8 T Cells with Shared Clusters in CD8 PBLs and TILs in Melanoma

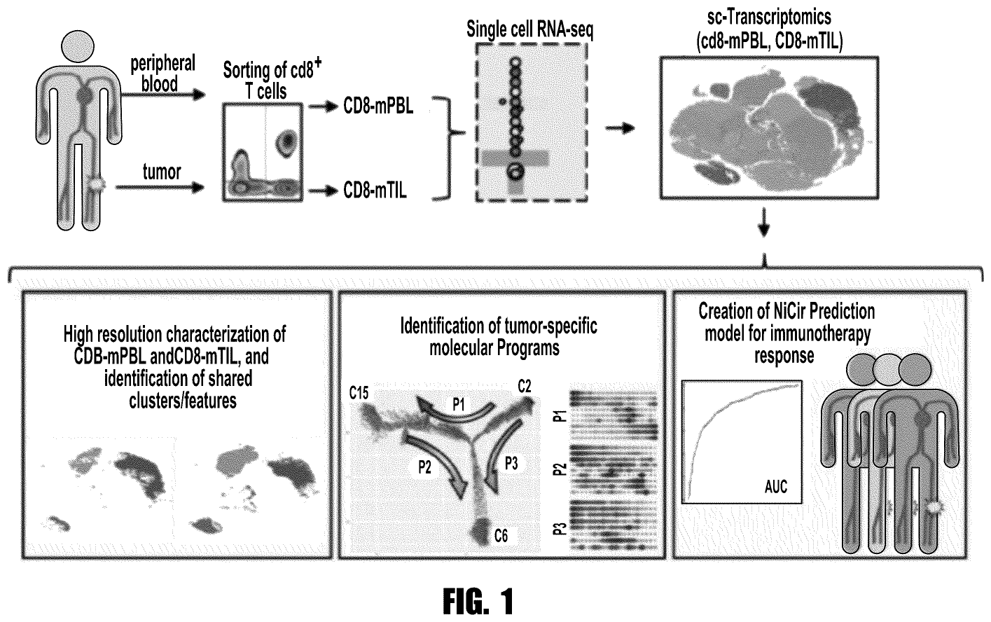

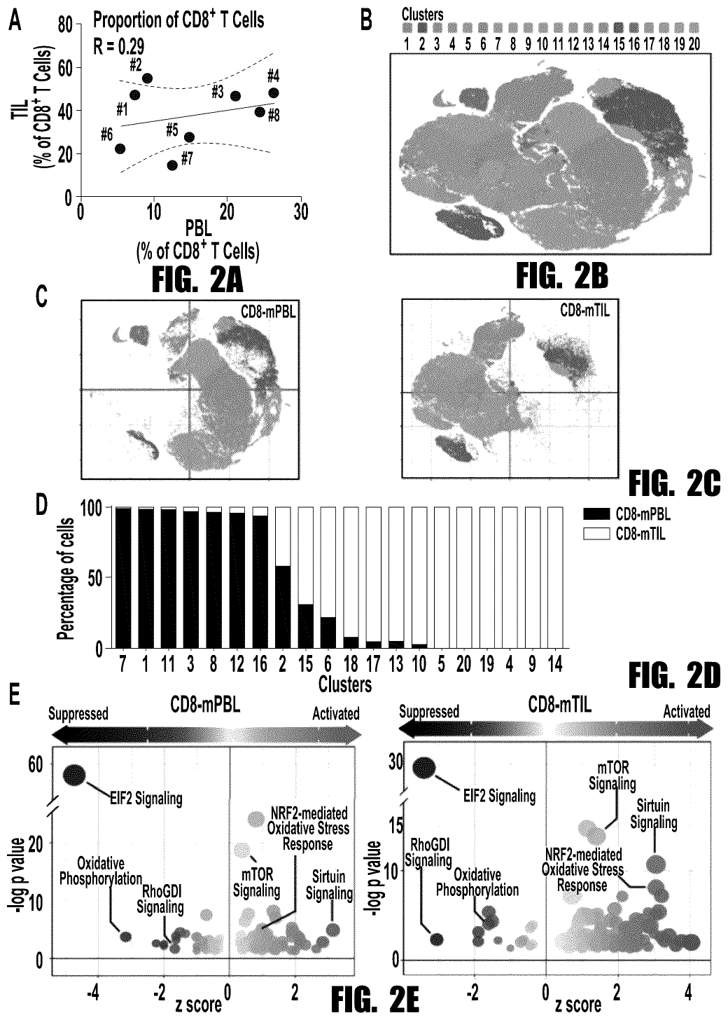

To develop a noninvasive method to predict effective immunotherapeutic responses, the inventors carefully characterized individual CD8 T cells from tumors and peripheral blood of melanoma patients. We performed single-cell RNA sequencing (scRNaseq) on isolated CD8 T cells from both melanoma PBLs (CD8-mPBLs) and TILs (CD8-mTILs) from eight advanced melanoma patients (FIG. 1). Compared with recent scRNaseq studies of melanoma (Sade-Feldman et al., Cell. 175:998-1013.e20 (2018)), their dataset was uniquely tailored to capture data from CD8 T cells with paired PBLs and TILs from the same patient. Through barcoding and combined analyses, we ensured matched processing and efficient downstream deconvolution of both samples. CD8-mPBLs and CD8-mTILs from the same patient were set as pairs and analyzed with flow cytometry for their proportion correlation analyses, and consistent with previous reports (Shao et al., PLOS One. 9:e102327 (2014)), the proportions of total CD8 T cells in PBLs were moderately correlated with those in TILs (R=0.29) among the examined patients (FIG. 2A).

Next, the inventors combined scRNaseq results of 173,061 CD8-mPBL/mTILs and performed transcriptomic analysis. Using t-distributed stochastic neighbor embedding (t-SNE) visualization, they uncovered a high level of heterogeneity of CD8 T cells in CD8-mPBL/mTIL, with a total of 20 clusters (clusters with <1% of cells in all samples were removed for further analysis; FIG. 2, B-D). Interestingly, both CD8-mTILs and CD8-mPBLs appeared activated in TCR signaling, components of the phosphatidylinositol 3-kinase pathway, CD28 and OX40 signaling, MHC II and NF-κB signaling, and the NRF2-mediated oxidative stress response (FIG. 2E), based on the significant differential expression of genes (false discovery rate [FDR]-adjusted t test, P<0.05) between CD8-mTILs and CD8-mTILs. In addition to enhanced pathways for T cell activation, CD8-mTILs exhibited traits of T cell exhaustion, stress responses, apoptosis, and suppressed immune checkpoint and PPAR signaling pathways (FIG. 2E). These observations are a testament to scRNaseq, showing that tumor-bearing conditions impart a systemic impact on circulating immune cells in addition to tumor-infiltrating cells. Bai et al., Sci. Rep. 5:13664 (2015).

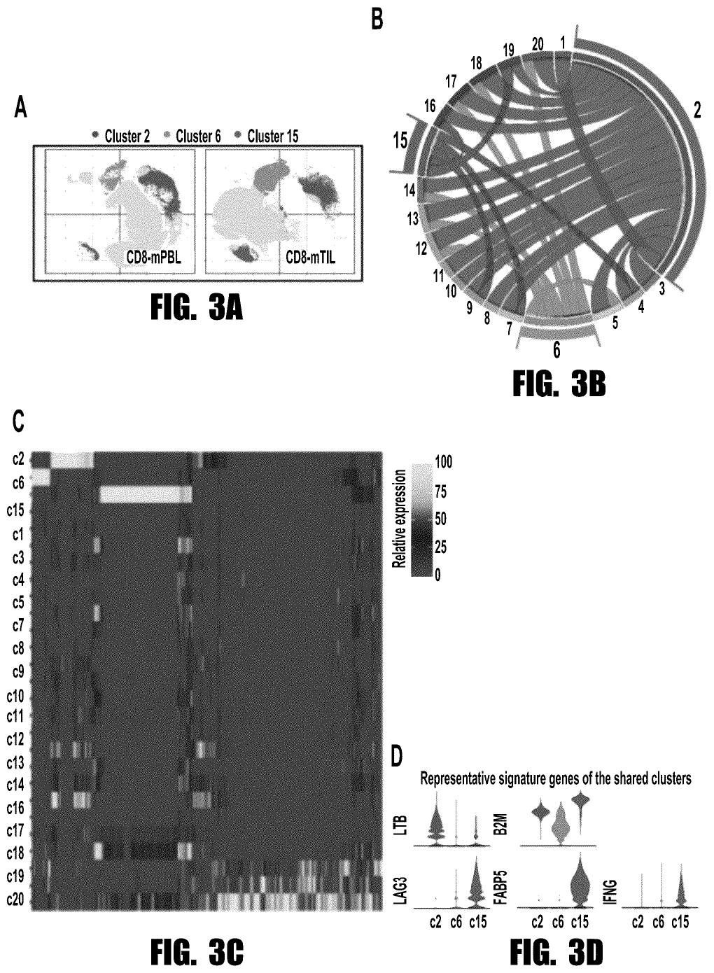

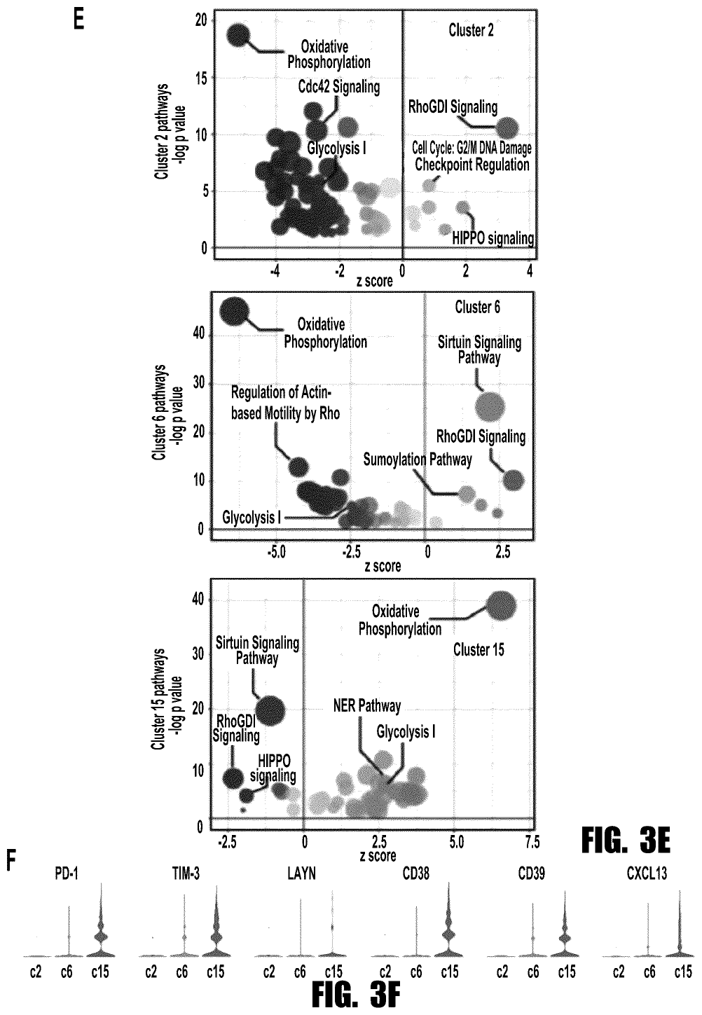

CD8-mPBLs and CD8-mTILs were quite distinct, with only three clusters shared between them (FIG. 2D and FIG. 3A). These three CD8-mPBL/mTIL shared clusters (clusters 2, 6, and 15) may permit tracking of CD8 T cells and help decipher their intracellular programming when exposed to the tumor environment, thus facilitating identification of important factors that dictate patient responsiveness to immunotherapy. To test this, the inventors first analyzed the transcriptomic correlation of clusters 2, 6, and 15 with all other clusters and found that cluster 2 showed similarity with most other clusters, with the exception of clusters 6 and 15 (FIG. 3B). Cluster 6 showed similarity with clusters 5, 12, 14, 18, and 20, whereas cluster 15 was similar to clusters 4, 9, 16, and 19. This nonoverlapping correlation of these three clusters supports their unique cellular programs and potential functions. Indeed, the 3D plot of all cells clearly depicts their distinct transcriptional profiles. Next, they extracted signature genes (FDR-adjusted t test, P<0.05) of the clusters (FIG. 3, C and D) that were found in all eight patients and performed a pathway analysis (FIG. 3E).

Cluster 2 expressed genes consistent with naive or resting T cell behavior, such as similar activation levels of DNA damage checkpoint regulation. Housekeeping RhoGDI (Rho GDP-dissociation inhibitor) pathways that are necessary to inhibit T cell activation were also highly active in cluster 2 cells (FIG. 3E). Burkhardt et al., Annu. Rev. Immunol. 26:233-259 (2008). Interestingly, HIPPO signaling was activated in cluster 2 (FIG. 3E), which includes the key inhibitor MOBIA, subunits of protein phosphatase, and 14-3-3, confirming the housekeeping function of these genes is consistent with that of naive or resting CD8 T cells. In addition, cluster 2 also contained suppressed Cdc42 signaling and T cell exhaustion signaling pathways that would ensure normal cell cycle and prevention of apoptosis.

Cluster 15 displayed pathway signatures mostly opposite to clusters 2 and 6, with prominent activation of OXPHOS, as well as activated glycolysis and NER (nucleotide excision repair) pathway (FIG. 3E). Contrary to clusters 2 and 6, inhibition of HIPPO and RhoGDI pathways were also observed in cluster 15 (FIG. 3E). Not surprisingly, T cell exhaustion markers were highly up-regulated in cluster 15, including the immune checkpoint molecules PD-1, TIM-3, LAG3 (FIG. 3, D and F), LAYN, and CXCL13 (FIG. 3F). Remarkably, ectonucleotidases CD38 and CD39, which are involved in NAD+ and ATP regulation, were also significantly up-regulated in cluster 15 (FIG. 3F). These characteristics of having not only expression of exhaustion markers but also clear metabolic activities add to our previous work of functionally suppressive CD8 T cells with high levels of checkpoints. Pfannenstiel et al., Cancer Immunol. Res. 7:510-525 (2019).

Compared with clusters 2 and 15, cells in cluster 6 were transcriptionally suppressed, with fewer than 100 genes (out of 11,000 total detected gene IDs in the whole dataset) up-regulated compared with all other clusters. Furthermore, most signaling pathways were inhibited or had low activation, and only a few pathways remained relatively active, such as the enhanced Sirtuin, RhoGDI, and sumoylation pathways. These analyses suggested that some CD8 T cells in mPBLs resemble CD8 T cells in mTILs, with three similar populations that are transcriptionally distinct from each other. To examine the potential bias effect of different numbers of T cells from different patients, we also analyzed our dataset with a t-SNE process using an equal number of cells from each patient. Consistently, the inventors not only observed comparable cluster distributions but also were able to identify the three PBL/TIL shared clusters with similar phenotypes and properties. Thus, theby show that that these three overlapping clusters are not due to an artifact of sample selection, a multidimensional reduction process, or a unique effect of a limited number of patients but are a phenomenon of the whole melanoma cohort in this study.

### Identification of Three Transciptomically Distinct Shared PBL/TIL Clusters

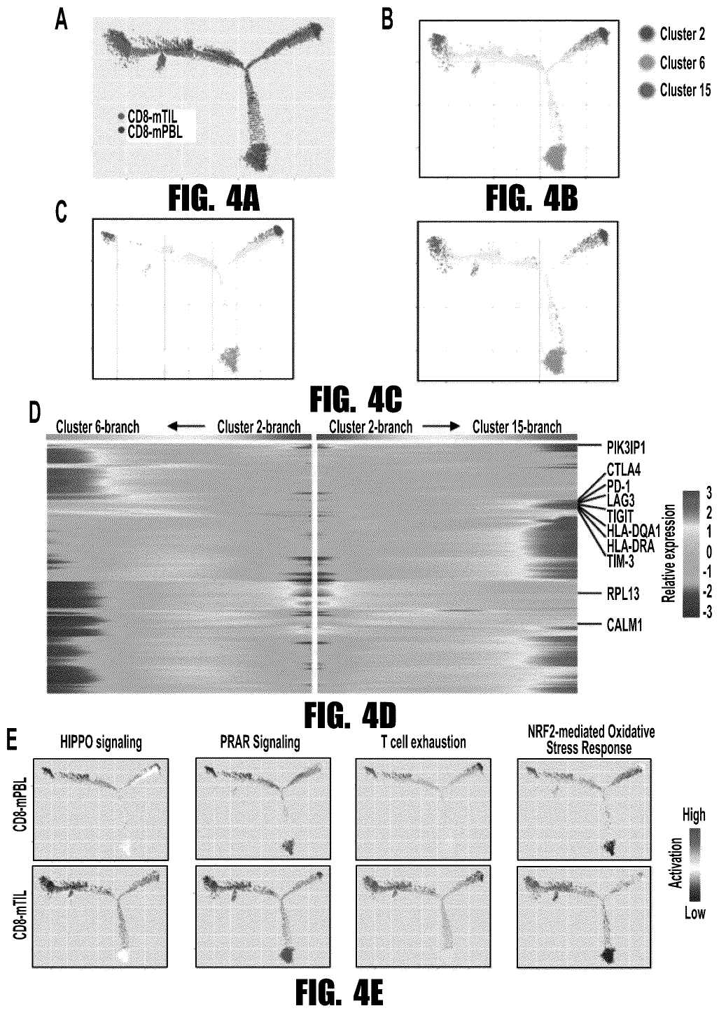

To further evaluate the cell state transition, the inventors randomly selected equal number of cells (1,000 cells from each sample) to perform the trajectory analysis using the Monocle package (Qiu et al., Nat. Methods. 14:309-315 (2017)) to reveal cell fate differentiation along the branch point trajectories. The overall distribution of CD8-mPBLs and CD8-mTILs formed a three-branched plot, with cluster 2, 6, or 15 at the tip of each branch (FIG. 4, A and B). Interestingly, CD8-mPBL continuously span from cluster 2 to cluster 15, with few isolated cells extending toward cluster 6, suggesting that cluster 2→15 is a tumor-specific pattern (FIG. 4, B and C). Further, CD8-mTILs are located along all three branches (cluster 2→15, cluster 2→6, and cluster 15→6), suggesting that CD8-mPBLs in cluster 6 are likely those migrating to and from tumors and the periphery. Since cell trajectory is calculated based on overall transcriptome profiles of each cell and assigned their relative position based on relative simulation, the location of clusters 2, 6, and 15 on the plot further confirms that these clusters represent the three most distinct transcriptomic patterns, suggesting three distinct function types. This adds to the importance of clusters 2, 6, and 15 as the only clusters that have overlapping populations between TILs and PBLs. Setting the branch of cluster 2 as root in Monocle, they tracked whole-transcriptome gene expression changes along the trajectory branches and identified gene clusters that were altered along the pseudotime progression from cluster 2→6 or cluster 2→15 (FIG. 4D). Interestingly, immune checkpoint genes, including CTLA4, PD-1, and LAG3, were highly expressed at the cluster 15 branch, whereas the PIK3IP1 gene, which is important for suppressing T cell activation, displayed an increased expression pattern in the cluster 2→6 direction (Uche et al., J. Exp. Med. 215:3165-3179 (2018)). In contrast, genes contributing to apoptosis and T cell exhaustion pathways (CALMI, HLA-DQA1, HLA-DRA, CTLA4, PD-1, LAG3, TIGIT, and TIM-3) were highly enriched in cluster 15, but not in cluster 2 or 6 (FIG. 4, D and E).

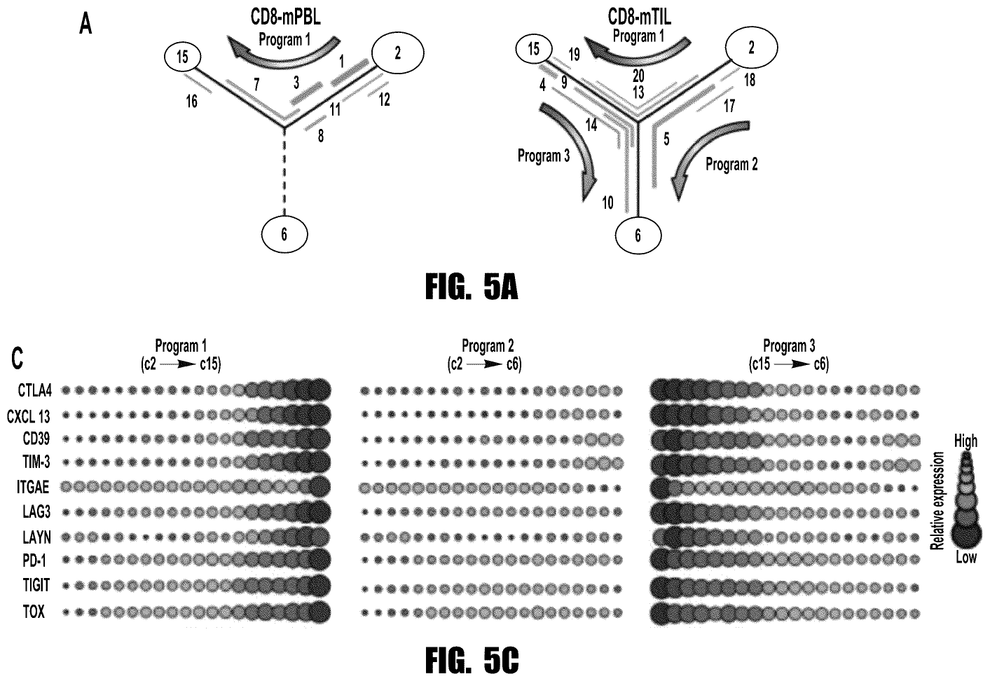

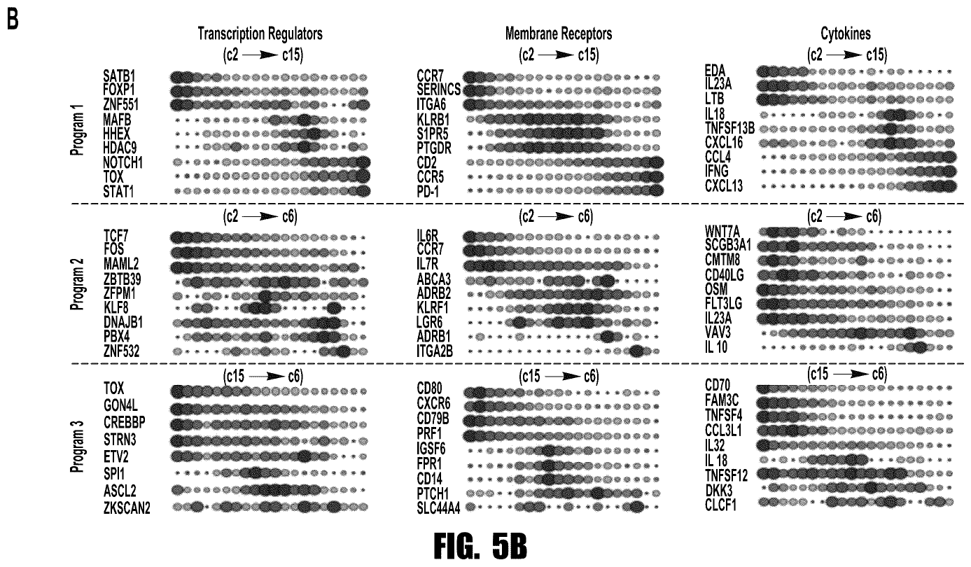

By pseudotemporal trajectory analysis, the inventors discovered three distinct programs that influence the gradual transition between the PBL/TIL shared clusters 2, 6, and 15 (FIG. 5 A). In CD8-mTILs, program 1 progressively transforms naive T cells (cluster 2) into a more “exhausted” CD8 T cell state (clusters 13, 20, and 19) along the trajectory and eventually transition to cluster 15 at the most distal of the trajectory branch (FIG. 5A). While program 2 drives the transition of naive CD8 T cells to cluster 6, program 3 transforms cluster 15 to cluster 6 (FIG. 5A). However, in CD8-mPBLs, only one program induces CD8 T cell transition, where program 1 is shown to trigger the transition of naive CD8 T cells (cluster 2) to cluster 15 (FIG. 5A). Of note, only program I significantly fosters the transition of naive CD8 T cells (cluster 2) into an exhausted CD8 T cell state (cluster 15) in both PBLs and TILs, with other clusters distributed along the program transitional states, indicating that CD8-mPBLs display certain molecular and gene profiles resembling CD8-mTILs (FIG. 5A).

Subsequently, they further analyzed the expression patterns of cell surface proteins and cytokines, which are vital in dictating CD8 T cell characteristics and functions in all the three programs (1, 2, and 3). Cytokines or secreted polypeptides that are known to maintain a resting T cell state, such as NOG and LTB, are enriched in cluster 2, whereas secreted molecules associated with T cell activation, such as CD40LG, CCL28, FLT3LG, TNFSF8, and NRG2, displayed an extended pattern along program 1 (cluster 2→15). Cytokines important for a strong inflammatory response in tumors are enriched at the cluster 15 end of program 1 (FIG. 5B). Further, dynamic changes of cell surface receptors also displayed similar patterns (FIG. 5B). Overall, cluster 15 CD8 T cells expressed high levels of genes for MHC II (HLA-DRB1, HLA-DKA, HLA-DPA1, and CD74), receptors for cell-to-cell interaction (CD2, CD47, CD27, IL2RG, and CXCR3), and dysfunctional markers and immune checkpoints such as PD-1, CTLA4, LAG3, and CXCL13. A recent review reported that CXCL13 was overexpressed in terminal dysfunctional CD8 T cells, suggesting a positive correlation between CXCL13 and dysfunctional anti-tumor efficacy (van der Leun et al., Nat. Rev. Cancer. 20:218-232 (2020)). Interestingly, IL6R, IL7R, and CCR7 were down-regulated from resting/naive cells (cluster 2) to cluster 15 CD8 T cells (FIG. 5B). More importantly, the inventors identified key immune checkpoint genes and transcription factors along the three program directions in CD8-mPBLs and CD8-mTILs (FIG. 5C). For example, in these programs, they identified TOX, which is a critical transcriptional factor that reprograms and drives CD8 T cells into an exhausted state during cancer progression and chronic infection. Mann and Kaech, Nat. Immunol. 20:1092-1094 (2019). It was found that TOX increases along program 1, along with multiple checkpoints, and is highest in cluster 15 CD8 T cells (FIG. 5 C).

Trajectory Analysis of PBL/TIL CD8 T Cells Illustrates how Metabolic Pathways like OXPHOS Differentiate Cluster 15 Compared With Classic Exhausted T Cells

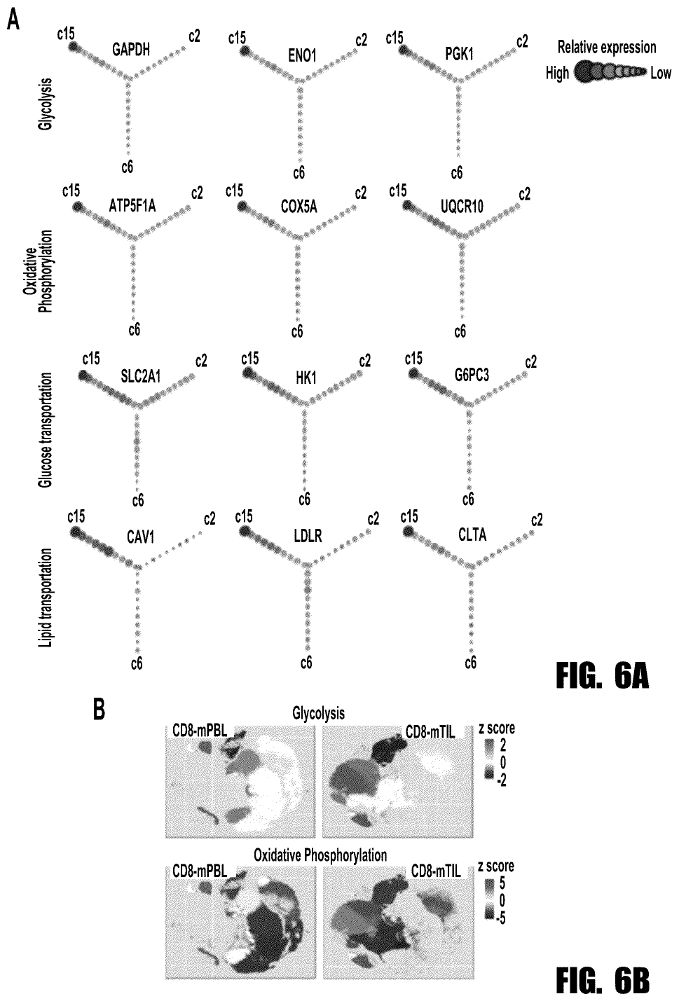

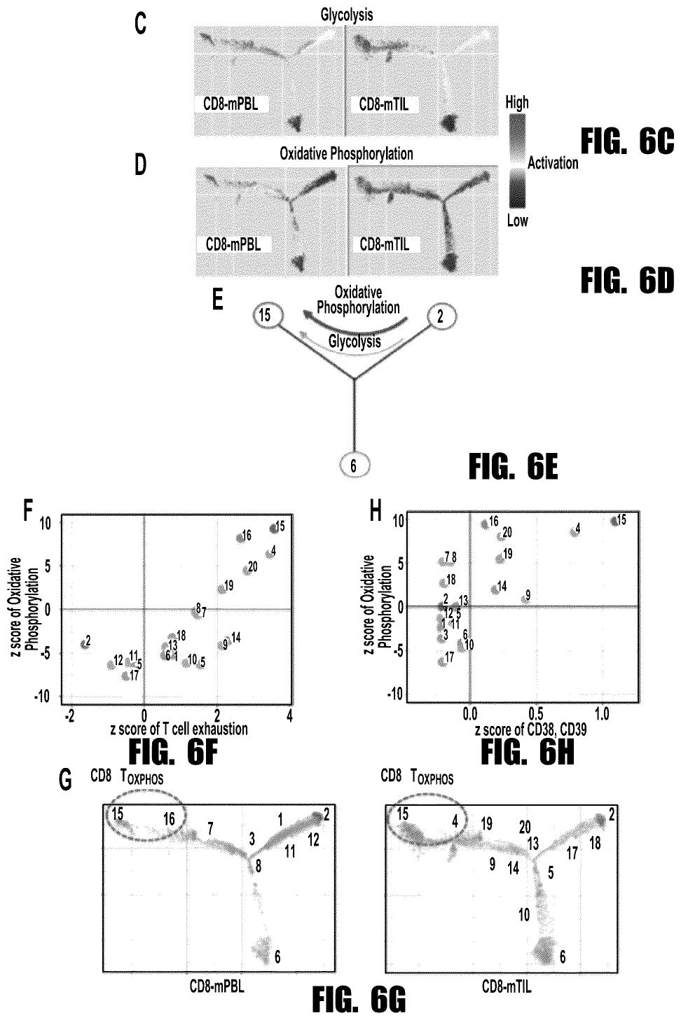

By transcriptomic analysis, the inventors next investigated the metabolic signaling in all clusters. Strikingly, they observed a clear pattern of cell metabolic shifting during cell transition from naive or effector state to a dysfunctional exhaustion state (FIG. 6). Among all clusters, cluster 15 displayed elevated OXPHOS, glycolysis, glucose, and lipid transportation activity (FIG. 6A). Genes involved in metabolic pathways, including OXPHOS, glycolysis, glucose, and lipid metabolism, were up-regulated in multiple clusters leading to cluster 15, such as PGK1, ATP5FIA, SLC2A1, and LDLR (FIG. 6A). In fact, cluster 15 had the highest cell metabolic rate, with the most pronounced activation of glycolysis and OXPHOS pathways (previously shown in FIG. 3E; see also FIG. 6, B-D). Remarkably, the OXPHOS pathway underwent a more significant change relative to glycolysis that enabled cells to transition from the state represented by cluster 2 to the state represented by cluster 15 (FIG. 6E). There is visible segregation of a high OXPHOS and glycolysis activation state in clusters 4 (TILs), 15 (shared PBLs/TILs), and 16 (PBLs) from the other clusters, with cluster 15 having the maximal activation of both OXPHOS and glycolysis.

Cluster 15, enriched with exhaustion markers (shown in FIG. 4D), has prominent levels of both OXPHOS and exhausted markers (FIG. 6F). Similar to cluster 15, we also observed high OXPHOS and exhaustion levels in both clusters 4 and 16 (FIG. 6F). On the other hand, clusters 14 and 9 in TILs have high exhaustion levels but low OXPHOS activity (FIG. 6F), thus indicating that they are classical exhausted TILs. Wherry and Kurachi, Nat. Rev. Immunol. 15:486-499 (2015). Commonly, exhausted T cells have low or mild expression of cytotoxic genes, which is very similar to clusters 4 and 9. However, it was unexpectedly observed that clusters 4, 15, and 16 are enriched with cytotoxic genes (e.g., PRF1 and GZMB); in particular, IFN-γ is elevated in cluster 15 (FIG. 3D), suggesting that these three clusters are distinctly different from classic exhausted T cells. Overall, the data demonstrate that clusters 4, 15, and 16 conglomerate into a unique T cell subset characterized by high exhaustion and OXPHOS states as well as increased cytotoxic gene expression (FIG. 6F).

The inventors next examined the biological progression of the various clusters based on their metabolic and transcriptional programs using pseudotime trajectory analysis. Consistent with the data shown in FIG. 3, they noticed a trifurcation of CD8 T cells into three distinct branches: resting/naive (cluster 2 branch, including clusters 1, 2, 12, 17, and 18), high OXPHOS with exhaustion markers (cluster 15 branch, including distal and related clusters 4, 15, and 16 [circled in FIG. 6G]), or inactive dormant (cluster 6) cells. Other clusters are distributed along the program transitional states (FIG. 6H). These results suggest that transcriptional programs in CD8-mPBLs have features similar to those found in CD8-mTILs, specifically in the formation of cluster 15, with clusters 4 and 16 having the closest proximity to cluster 15 (FIG. 6H).

To functionally identify these unique subset of T cells, the inventors looked for potential cell surface markers that could differentiate the transcriptionally related clusters 4, 15, and 16 from other clusters. By trajectory plot, they observed a gradual enrichment of PD-1, CD38, and CD39 expression leading toward cluster 15, inferring that these markers could be used for stratification. It was found that both CD38 and CD39 are able to distinguish clusters 4 and 15 from other clusters, but not in cluster 16, which is found in PBLs (FIG. 6H). Consistently, clusters 4 (TILs) and 15 (shared PBLs and TILs) also have high levels of combined expression of PD-1 and CD38/CD39, but not cluster 16, which may have to do with not being under the direct influence of the tumor microenvironment. Together, clusters 4, 15 and 16 form a unique spectrum of CD8 T cells defined transcriptionally by their distinct features of high OXPHOS, exhaustion (e.g., PD-1), and cytotoxic gene expression, which the inventors coin CD8+ TOXPHOS. However, for cellular-level evaluation, to validate these transcriptional findings, the ability to identify PD-1, CD38, and CD39 expression will at least allow for studies of CD8+ TOXPHOS clusters 4 and 15.

### High-Bioenergy CD8 T Cells in Refractory Melanoma Patients

The inventors reported that many melanoma patients have a significant number of CD8 TILs expressing high levels of immune checkpoints and that these same cells have active immune suppression of autologous healthy lymphocytes. Pfannenstiel et al., Cancer Immunol. Res. 7:510-525 (2019). In their single-cell transcriptomic analysis, they found that CD8+ TOXPHOS cells have the strongest metabolic signal within CD8 TILs. These findings led the inventors to explore whether purified CD8 TILs from patients enriched with CD8 T cell subsets expressing high levels of immune checkpoints have a metabolic rate. Due to the limitations of the Seahorse glucose oxidation assay, which requires a large number of cells, they did a simple comparison as a starting point with healthy T cells.

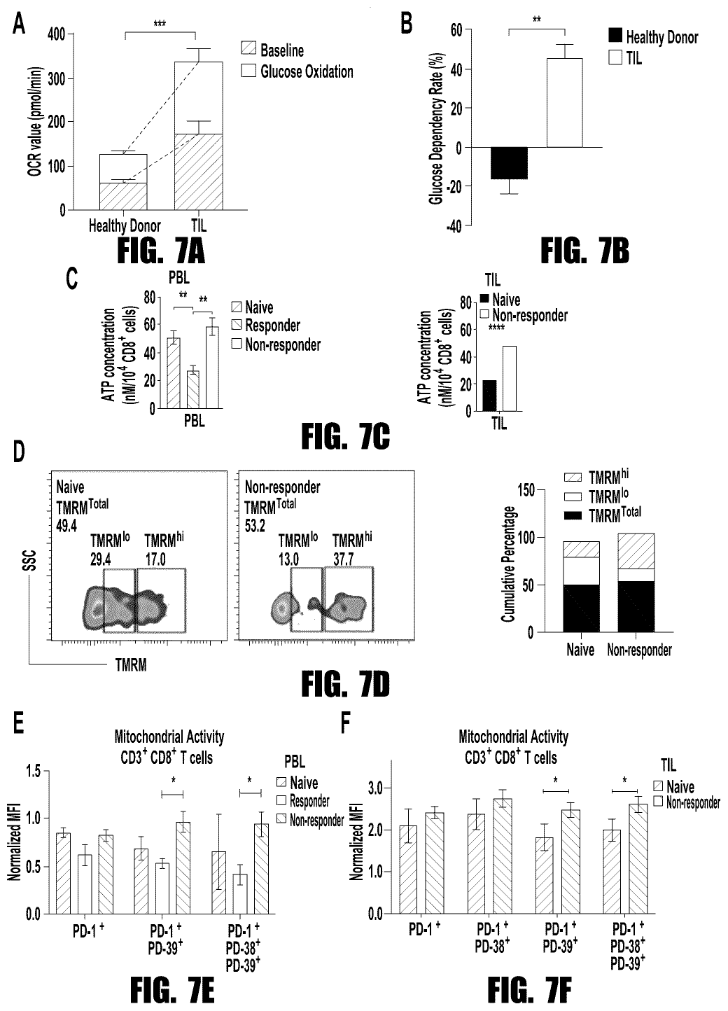

They first performed the Seahorse assay to validate the involvement of glucose oxidation during glycolysis by determining the oxygen consumption rate (OCR) of healthy CD8 PBLs and CD8+ PD-1+ TILs (FIG. 6A shows that CD8+ TOXPHOS cells account for the largest signal of glucose transportation in melanoma patients' CD8 T cells). Melanoma CD8 TILs were indeed fueled by glucose oxidation as validated by higher OCR compared with healthy PBLs (FIG. 7A). Of note, these CD8 TILs not only depended on glucose oxidation but also had high initial OCRs (FIG. 7A). Interestingly, CD8 TILs had strong dependency on oxidation of glucose, as demonstrated by increased glucose dependency rate (FIG. 7B). Glycolysis, in concert with OXPHOS, generates energy through ATP production. Thus, ATP production was measured in TILs and PBLs of melanoma patients. Using an ATP assay, they validated that peripheral CD8 T cells of ICI-nonresponder melanoma patients produced significantly higher ATP as compared with responders (FIG. 7C), although they do note that the changes are not as striking in PBLs (P<0.01) compared with TILs (P<0.0001). The inventors biobank of surgical specimens contains TILs mainly from two sources: (1) ICI nonresponders and (2) patients who have not yet undergone systemic therapy (naive). CD8 TILs from nonresponders produced significantly higher ATP compared with naive patients (FIG. 7C). The reduction of differential membrane potential is equivalent to a reduced capacity for ATP production and other related mitochondria functions. Hence, to answer whether these high-bioenergy-state CD8 T cells were generated by the CD8+ TOXPHOS cell subpopulation within them, the inventors performed flow cytometry combined with metabolic analyses using tetramethylrhodamine methyl ester (TMRM), a fluorescent dye that accumulates in the functional mitochondria caused by differential membrane potential, as a marker of OXPHOS. Davis et al., Nat. Cell Biol. 22:310-320 (2020). PBL and TIL CD8 T cells were co-stained with enriched-surface markers identified in their scRNaseq analysis, including PD-1, CD38, and CD39, and MitoTracker to determine the mitochondrial membrane potential relative to mitochondrial total mass.

Although expression of CD38 and CD39 in CD8+ PD-1+ T cells does help characterize the CD8+ TOXPHOS cells, additional transcriptional assays are required for exact determination of this immune population. The inventors compared CD8+ TOXPHOS cells to other CD8 subpopulations; they expressed a unique transcriptomic signature, even when compared with effector and central memory cells as well as exhausted CD8 T cells. Still, within this PD-1+subgroup (that have high CD38/39), we observed increased levels of OXPHOS (FIG. 7D), consistent with our CD8+ TOXPHOS cells designation, which is distinct from the classically defined PD-1+exhausted T cells, which have been shown to have impaired mitochondrial function. Vardhana et al., Nat. Immunol. 21:1022-1033 (2020). Within this population of CD8+ PD-1+CD38+CD39+ T cells, they illustrate two levels of TMRM: (1) high TMRM (TMRMhi) and (2) low TMRM (TMRMlo; for a schematic representation of flow cytometry in TILs, see FIG. 7 D). Non-responder patients mainly have TMRMhi and negligible TMRMlo levels, while naive patients have higher TMRMlo but lower TMRMhi in TILs (FIG. 7D). Although non-responders have higher TMRMtotal in PBLs, no obvious difference was observed in CD8+ PD-1+CD39+ and CD8+ PD-1+CD38+CD39+ TILs. TMRMlo in CD8+ PD-1+CD39+ and CD8+ PD-1+CD38+CD39+ T cells in both PBLs and TILs do not stratify for non-responders. Most importantly, significantly higher mitochondrial membrane potential was observed in PBL CD8+ PD-1+CD39+ and CD8+ PD-1+CD38+CD39+ T cells of non-responders of melanoma patients (FIG. 7E). However, CD38 as a marker did not correlate with high mitochondrial membrane potential in PBLs. In TILs, non-responders have elevated mitochondrial membrane potential in both CD8+ PD-1+CD39+ and CD8+ PD-1+CD38+CD39+ cells, but not in CD8+ PD-1+CD38+ T cells, compared with naive melanoma patients (FIG. 7F).

Importantly, the inventors' data suggest the high possibility of stratifying responders and non-responders using PBL-based metabolic activity assays (ATP and TMRM). However, they did not observe significant stratification between patients before therapy (naive) and after failed ICI therapy (non-responders). The likely reason is that the naive group contains both responders and non-responders, masking the stratifying effect. Further, these findings highlight the possibility that these metabolic pathways are not only phenotypic but also functional. Therefore, targeting them to modulate CD8+ TOXPHOS cells could be a novel way to improve ICI therapy.

### High-Accuracy Predictive Model for Immunotherapy Response

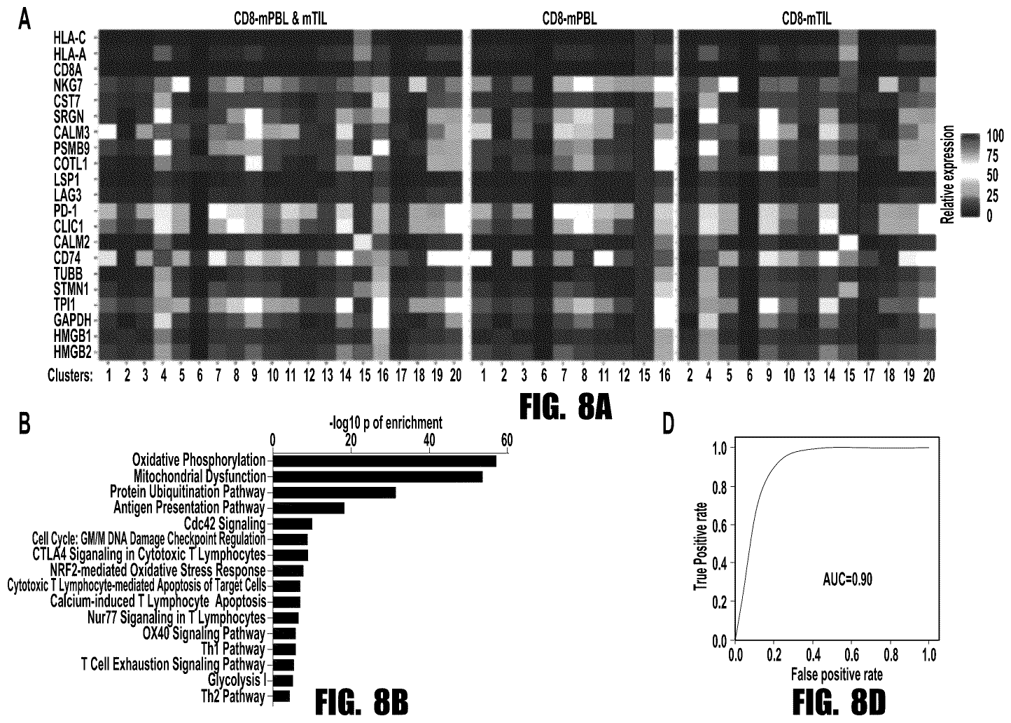

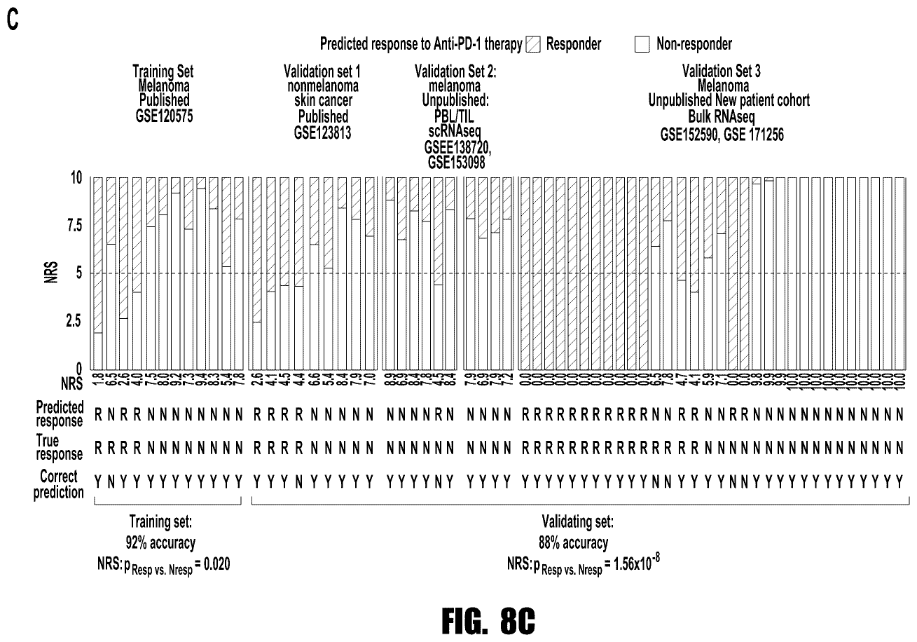

Based on the association between CD8+ TOXPHOS cells and ICI resistance, the inventors further developed a predictive model of immunotherapy responses using signature genes enriched in cluster 15-like cells, which include not only clusters 4 and 15 but also PBL cluster 16. To screen for a predictive immunotherapy response gene signature, they first performed whole-transcriptome gene coexpression analyses in the three unique PBL/TIL shared populations (clusters 2, 6, and 15). Genes that displayed significant positive correlation with PD-1 expression were highly expressed in clusters 4 and 16 but were most pronounced in cluster 15 (FIG. 8A). By gene ontology analyses, genes coexpressed with PD-1 in CD8+ TOXPHOS cell clusters were enhanced in the mitochondrial dysfunction pathway, Cdc42 signaling, and CTLA4 signaling (FIG. 8B). Interestingly, OXPHOS was the most activated pathway in cluster 15-like cells, further substantiating the importance of immune metabolism in checkpoint-based immunotherapy.

To develop a GEP predictive of immunotherapy response, the inventors selected the most significantly elevated top 20 coexpressed with PD-1 (FIG. 8A). One of these is the immune checkpoint LAG3, which binds to MHC II and is associated with exhausted CD8 T cells in human tumors. Chen and Mellman, Nature. 541:321-330 (2017). Another, GAPDH, a key enzyme of glucose metabolism, was overexpressed in CD8+ TOXPHOS cells. They also identified several genes less known for their roles in cancer ICI therapy (including serglycin, a small proteoglycan important for cytotoxic T cell secretory function, and cystatin F [CST7], a cysteine peptidase inhibitor) that may regulate immune cell function in tumors. Perišić Nanut et al., Front. Immunol. 8:1459 (2017). Besides CST7, CD74 and HLA genes (HLA-A and HLA-C) known to be crucial for CD8 T cell function were also found to be up-regulated in both CD8 PBLs and TILs with high PD-1 coexpression.

Using the 21-gene GEP (including PD-1), the inventors built a logistic regression model using a training cohort from a published study with scRNaseq dataset of TILs from melanoma patients undergoing immunotherapy (GSE120575; Sade-Feldman et al., Cell. 175:998-1013.e20 (2018)). Their model is designed to predict the status of each T cell in a patient. Then, the proportion of nonresponding cells in each patient was rescaled to 0˜10, depicting the nonresponse score (NRS). A median NRS of 5 was selected as the threshold score, and patients with an NRS>5.0 were considered non-responders. Using the training dataset, the inventors predicted the ICI response in 11 of the 12 patients, yielding an accuracy of 92%, with non-responders having significantly higher NRSs than responders (t test, P=0.02; FIG. 8C).

To further validate our model, the inventors applied it to four additional datasets, including one published dataset and three independent validation patient cohorts from their institution. Due to the limited availability of clinical immunotherapy response data from public repositories, they validated their model using a published scRNaseq dataset of TILs from nonmelanoma skin cancer patients receiving anti-PD-1 immunotherapy with only one prediction error (GSE123813; dataset 1; FIG. 8C; Yost et al., Nat. Med. 25:1251-1259 (2019)). Next, they went on to further examine the accuracy of their model using patient cohorts from their institution. The inventors first employed scRNaseq of CD8+ PD-1+ T cells from five non-responder patients with matched PBLs-TILs (dataset 2: five PBLs and five TILs; note three of these patients [six samples, paired TILs-PBLs] were from our original discovery set [GSE138720]), and then they performed scRNaseq on two more refractory patients' TIL and PBL CD8 T cells (GSE153098). In addition, they tested the model using a bulk RNA sequencing (RNaseq) platform. Based on their power calculation, 14 blood samples are required to achieve a power of 0.9. To ensure they achieved this, they collected and performed bulk RNaseq on a total of 32 blood samples (15 responders and 17 non-responders) from melanoma patients (dataset 3; FIG. 8C) and four lung cancer patients (dataset 4) treated with immunotherapy. Remarkably, combining analysis of validation sets 1-3, their model achieves 88% accuracy (note, in the melanoma, it achieved accuracy of 89% in set 2 and 88% in set 3, FIG. 8C). Surprisingly, it achieves 100% accuracy in a small cohort of lung cancer patients (not included in the overall accuracy calculation of melanoma). These results validate their GEP model as an efficient predictor of immunotherapy responses; this algorithm is effective whether the source material is from the tumor or obtained less invasively using blood-based approaches. In all of the validation cohorts, their model assigned significantly higher NRSs to nonresponders than responders (t test, P=1.56×10⁻⁸; FIG. 8C) and effectively discriminated them with an area under the receiver-operating characteristic curve (ROC-AUC) of 0.90 (FIG. 8D). Given that blood-based approaches have greater potential for utility in oncology, as they do not require invasive acquisition of CD8 TILs, the inventors termed their prediction platform, for simplicity, the noninvasive circulating T cells model (NiCir). Collectively, the validation data demonstrate that NiCir has high predictive accuracy in TILs or PBLs and can be applied to multiplatform datasets. Of note, given NiCir's ability to predict ICI responses from peripheral blood CD8 T cells, it can now be used as a noninvasive tool to predict immunotherapy responses in the clinical setting.

### Discussion

For a number of years, the inventors' group has defined tumor-induced dysfunctional changes in infiltrating T cells. Their recent work showed that in cancers like melanoma, a large percentage of tumor-associated CD8 T cells express high levels of checkpoints like PD-1, and these same cells can actively suppress other immune effectors. Pfannenstiel et al., Cancer Immunol. Res. 7:510-525 (2019). Thus, expressing classical exhaustion markers (e.g., checkpoints) does not necessarily define a CD8 T cell as anergic and can be associated with a form of active dysfunction; little is known about this phenomenon outside of nonmalignant pathologies. Flippe et al., Immunol. Rev. 292:209-224 (2019). Instead, targeting suppressor cells like CD4 T regulatory cells, myeloid-derived suppressor cells, and M2-macrophages remains the main focus in the majority of cancer studies. Liu et al., Immunother. 67:1181-1195 (2018). CD8 T cells, a critical player in immunotherapy effectiveness, have been extensively studied, but mainly in terms of their exhaustiveness or inefficiency of tumor infiltration. Thus, if additional TIL CD8 T cell heterogeneity exists within the population targeted by checkpoint inhibitors, then this should prompt additional investigation to improve this line of immunotherapeutics.

Interestingly, CD8+ PD-1+ T cells in the periphery of cancer patients were found to contain overlapping TCRs with CD8 T cells in the tumor and are also the most likely to yield (after expansion and adoptive transfer) an anti-tumor response. The NCI surgery branch that made this discovery surmised that the peripheral and intratumoral TCR sharing CD8+ PD-1+ T cells could be interrelated. Gros et al., Nat. Med. 22:433-438 (2016). If this peripheral blood-intratumoral relationship exists, then it may provide insight into the same tumor-derived CD8 T cells expressing checkpoints that the inventors have been studying and were found to be affected by ICIs. The inventors hypothesized that they can leverage this PBL-TIL relationship to gauge the status of immunotherapy resistance and learn from the related biochemical pathways within CD8 T cell subpopulations.

Using scRNaseq, the inventors discovered that only three unique transcriptionally shared/overlapping clusters in PBL-CD8 and TIL-CD8 T cells exist, two of which they determined spawn from naive CD8 T cells through specific reprogramming, as illustrated in their trajectory analysis; they defined them above as inert dormant and CD8+ TOXPHOS cells. Unlike classic exhausted CD8 T cells, CD8+ TOXPHOS cells in particular appear to contain many of the hallmarks of exhausted CD8 T cells that they previously studied, including checkpoint expression (e.g., PD-1), but they also expressed cytotoxic markers (e.g., IFN-γ and GZMB), corroborating recent reports illustrating that exhausted T cells are not homogeneous but a gradual transition. Yost et al., Nat. Med. 25:1251-1259 (2019). This is not completely surprising, as the inventors and others had already identified that suppressor CD8 T cells (which they later found express multiple checkpoints) regulate other cells via expression of IFN-γ. Robb et al., Blood. 118:3399-3409 (2011). In fact, studies show that IFN-γ, depending on the conditions, can act as an effector or suppressor immune mediator, and the ramifications of their findings are under investigation. Lee and Ashkar, Immunol. 9:2061 (2018). These CD8+ TOXPHOS cells also have up-regulated metabolic pathways, consistent with the fact that dysfunctional CD8 T cells are functionally diverse. van der Leun et al., Nat. Rev. Cancer. 20:218-232 (2020). The inventors' discovery of metabolically active CD8+ TOXPHOS cells supports recent work that CD8 T cells in TILs with PD-1 expression have two distinct states, low and high metabolism. Hartmann et al., Nat. Biotechnol. 39:186-197 (2021). Interestingly, in their study, which used CYTOF (cytometry by time of flight), Hartmann et al. found that cells that, based on the inventors' work, would likely be CD8+ TOXPHOS cells interface directly with the tumor, whereas the more classical exhausted CD8 T cells were found in the peripheral areas of the tumor, thus supporting a unique role for CD8+ TOXPHOS cells in tumor immunology.

Recent scRNAseq studies have shown that TIL CD8 T cells with hallmarks of exhaustion (e.g., expression of PD-1) contain subpopulations that are actively cycling and proliferating. Li et al., Cell. 176:775-789.e18 (2019). However, few studies have focused on identifying the mechanistic drivers of these active dysfunctional cells. Most reports study lymphocytes in general and not solely lymphocytes on CD8 T cells. Despite recent attempts by using scRNaseq to investigate and identify CD8 T cells in the peripheral blood and TILs, only Wu et al. and Fairfax et al. have reported the identification of shared populations (and only within cytotoxic CD8 T cells; they did not identify the CD8+ TOXPHOS cells found in the inventors study. Fairfax et al., Nat. Med. 26:193-199 (2020); Wu et al., Nature. 579:274-278 (2020). By focusing on enriched CD8 T cells in both TILs and PBLs, with sufficient cell numbers from individualized paired patient samples, the inventors' had greater sensitivity and resolution compared with most previous studies, allowing them to better analyze these underexplored CD8 subpopulations.

Recent studies have explored the role of TIL T cell metabolism in regulating immunotherapy, where metabolic properties of T cells exert an essential role in anti-tumor immunity. Hartmann et al., Nat. Biotechnol. 39:186-197 (2021). The metabolic state of peripheral blood CD8 T cells is an even more immature field. Voss et al., Nat. Rev. Immunol. 21:637-652 (2021). Remarkably, the inventors' data indicate that higher metabolic activity, including up-regulation of OXPHOS and glycolysis within CD8+ TOXPHOS cells in TILs and PBLs, is associated with ICI resistance. To translate this work for both potential immunotherapeutic targeting and cellular validation, they identified uniquely high expression of the cell surface molecules CD38 and CD39 on CD8+ TOXPHOS cells, whose inhibitors are being assessed in current ongoing clinical trials targeting solid cancers like melanoma. van der Leun et al., Nat. Rev. Cancer. 20:218-232 (2020). Both CD38 and CD39 are ectonucleotidases that play a role in NAD+/ATP regulation, which is connected to several metabolic pathways. Based on these findings, it is possible that these ectonucleotidases are associated with the high metabolic state that the inventors observed in CD8+ TOXPHOS cells.

Another group demonstrated that activation of PBL Ki-67+PD-1+CD8+ T cells (where Ki-67 represents increased proliferation capacity and assumedly increased metabolism) before anti-PD-1 therapy correlates with poor ICI response. Huang et al., Nature. 545:60-65 (2017). The same group later reported that patients with sustained high levels of Ki-67 in PD-1+CD8+ T cells in blood have better outcomes after a single dose of neoadjuvant anti-PD-1 therapy. Huang et al., Nat. Med. 25:454-461 (2019). To understand this discrepancy, they hypothesized that these proliferating T cells likely convert to a more reinvigorated and functional state after treatment with anti-PD-1 therapy. Whether these cells in PBLs represent CD8+ TOXPHOS cells or other populations that can contain these makers is unknown. However, if these Ki-67+PD-1+CD8+ exhausted T cells are CD8+ TOXPHOS cells, then it would imply that patients whose CD8+ TOXPHOS cells become reinvigorated by anti-PD-1 therapy have better outcomes, and identifying why this does not occur ubiquitously could create strategies to improve survival in all melanoma patients. The inventors study, which illuminates many of the mechanisms found to be activated in CD8+ TOXPHOS cells, offers many targets that may become new therapeutic avenues.

Anti-PD-1-based ICI has revolutionized the care of melanoma; unfortunately, a correlative assay that reliably aids clinicians in predicting who will respond continues to be an unmet need . Currently, only three predictive biomarkers were approved by the US Food and Drug Administration for ICI therapies for any cancer: protein expression of PD-L1 by immunohistochemistry, microsatellite instability status by PCR phenotyping, and tumor mutation burden. Boyiadzis et al., J. Immunother. Cancer. 6:35 (2018); Davis and Patel, J. Immunother. Cancer. 7:278 (2019); Prasad and Addeo, No. Ann. Oncol. 31:1112-1114 (2020). In recent years, most biomarker exploration has been focused on searching for methods that use minimally invasive approaches. To date, despite a large amount of effort, whether it be with tumor tissue or PBLs, and using various “omic” technologies or immunohistochemistry-based studies, there is no standard correlative assay used for ICI therapy in the treatment of melanoma.

Based on the inventors' discovery set, they developed a GEP-coined NiCir that they validated with a published dataset and four independent datasets, including from TILs and PBLs from their own patients. NiCir had an average accuracy of 88% in the validation datasets, indicating good prediction value across a spectrum of different sequencing platforms. NiCir showed excellent predictive power, with an area under the curve (AUC) of 0.90, as compared with several previously reported immunotherapy response predictions; for example, IMPRES (immunopredictive score) achieved an AUC of 0.83 for overall accuracy (Auslander et al., Nat. Med. 24:1545-1549 (2018); a radiographical characteristic in non-small cell lung cancer patients using artificial intelligence with an AUC of up to 0.76; and tumor mutational burden in non-small cell lung cancer predicting ICI response with an AUC range of 0.554-0.755. The inventors plan to translate this work into an assay that can be easily applied in a clinical setting for more timely decision making.

In summary, the results increase the field's knowledge of the heterogeneity of dysfunctional CD8 T cells in cancer patients. The work implicates the potential of manipulating the immunometabolism of T cell populations like CD8+ TOXPHOS cells to regulate and improve anti-tumor immunity. Moreover, the presence of CD8+ TOXPHOS cells in particular can be exploited even with blood-based approaches for a rapid predictive ICI efficacy assay to improve clinical outcomes in melanoma patients.

### Materials and methods

### Study Approval

All human tissue was obtained at the Cleveland Clinic under a protocol approved by Cleveland Clinic's institutional review board, and written informed consent was obtained from each patient. All patient samples used for scRNaseq were immunotherapy non-responders. Peripheral blood samples of immunotherapy responders were used as additional validation cohort for the predictive model (NiCir).

### Isolation of PBLs and TILs

Peripheral blood mononuclear cells were purified from buffy coats by centrifugation over a Ficoll-Hypaque gradient according to the manufacturer's protocol (GE Healthcare). Matched PBLs and tumor specimens were obtained from patients with melanoma. PBLs were obtained by venipuncture and isolated by centrifugation over a Ficoll-Hypaque gradient. After surgical resection, tumor specimens were rinsed with antibiotic-containing media and minced with crossed scalpels under sterile conditions. Enzymatic digestion was then used to dissociate tumor tissue using 1,500 U/ml collagenase IV (Gibco/Life Technologies), 1,000 U/ml hyaluronidase (Sigma), and 0.05 mU/ml DNase IV (Gibco) in RPMI for 1 h at 37° C. followed by mechanical agitation. The resulting single-cell suspensions were separated from debris by centrifugation over a Ficoll-Hypaque gradient followed by being immediately gated and sorted for LIVE>CD3+>CD8+ using LIVE/DEAD (Invitrogen; #L10119) CD3 (BioLegend; #300406) and CD8 (BioLegend #300912). The negative population was dumped using dump channel PE-Cy7 (YG) for CD4 (BioLegend; #300454), CD19 (BioLegend; #302215), and CD56 (BioLegend; #302215). On average, sorted cell counts were between 18,605 and 297,000, with an average of 91% efficiency for scRNaseq. Sorted cells were analyzed with flow cytometry to ensure over 90% purity.

Seahorse assay

The oxygen consumption and glucose dependency of healthy donor PBLs and melanoma TILs were determined using the Seahorse XF Mito Fuel Flex Test (Agilent; 103260-100) with the XFe96 Bioanalyzer (Agilent) according to the manufacturer's instructions. Briefly, enriched CD8 T cells of PBLs and sorted CD8+ PD-1+ T cell TILs were plated into Agilent Seahorse XF96 microplate in triplicate (75,000 cells/well) overnight, followed by 1-h incubation at 37° C. in a non-CO₂ incubator on the day of the assay. The pyruvate inhibitor UK5099 (2 mM) was injected into the microplates to determine the glucose oxidation dependency. Baseline OCR was monitored at 24 min, followed by sequential pyruvate inhibitor UK5099 (2 mM) injections, and the OCR readings were recorded for a duration of 2 h at 8-min intervals. Glucose OCR and dependency were calculated using the precent dependency equation based on the instructions, and graphs were plotted with GraphPad Prism software.

### ATP Assay

The ATP bioluminescence assay was used to quantify ATP levels. TIL and PBL samples were labeled with CD8 microbeads (Miltenyi Biotec; #130-045-201, MACS) and then positively selected for CD8 T cells by passing them through the LS column (Miltenyi Biotec; #130-042-401, MACS) according to the manufacturer's instructions. ATP levels of the positive-selection CD8 T cells were determined using a Luminescent ATP Detection Assay kit (Abcam; ab113849) following the manufacturer's instructions. Briefly, CD8 T cells were incubated with detergent for 5 min, followed by incubation with substrate solution for 10 min at room temperature in the dark with agitation. Then, luminescence was quantified on a microplate reader (Molecular Devices; SpectraMax M2). The luminescence values of ATP standards were determined in a similar manner. ATP concentration was calculated according to an ATP-standard curve.

### Flow Cytometry

Cell viability was determined using LIVE/DEAD Fixable DEAD Cells Stain Kit (Thermo Fisher Scientific). Human PBLs (naive, n=4; responders, n=5; non-responders, n=7) and TILs (naive, n=5; non-responders, n=6) from melanoma patients were stained with CD3, CD8, PD-1, CD38, CD39 (all from BioLegend), TMRM (Thermo Fisher Scientific), and MitoTracker Green (Thermo Fisher Scientific). Cells were incubated for 20 min at 37° C. with TMRM and MitoTracker Green according to the manufacturer's instructions. Next, the cells were stained with antibodies for 13 min at 4° C. in FACS buffer, followed by flow cytometry analysis. Compensation controls and fluorescence minus one controls were used to determined cell populations. Flow cytometry was performed using a BD LSRFortessa and analyzed with FlowJo software, and normalized mean fluorescence intensity (MFI) graphs were plotted with GraphPad Prism software.

scRNaseq Library Preparation and Data Processing

All cells were resuspended in DPBS with 0.04% BSA, and immediately processed for scRNaseq as follows. Cell count and viability were determined using trypan blue on a Countess FL II, and ˜12,000 cells were loaded for capture onto the Chromium system using the v2 single-cell reagent kit according to the manufacturer's protocol (10× Genomics). Following capture and lysis, cDNA was synthesized and amplified (12 cycles) as per the manufacturer's protocol (10× Genomics). The amplified cDNA from each channel of the Chromium system was used to construct an Illumina sequencing library and sequenced on an Illumina NovaSeq with 150-cycle sequencing (asymmetric reads per 10× Genomics). Illumina basecall files (*.bcl) were converted to FASTQs using CellRanger v3.0, which uses bc12fastq for FASTQ file generation. FASTQ files were then aligned to GRCh38 human reference genome and transcriptome using the CellRanger v3.0 software pipeline with default parameters as reported previously (Larkin et al., 2019); this demultiplexes the samples and generates a gene versus cell expression matrix based on the barcodes and assigns unique molecular identifiers (UMIs) that enable determination of the individual cell from which the RNA molecule originated.

### Bulk RNaseq Library Preparation and Data Processing

PBLs were sorted for CD8 T cells using a selective gating strategy based on staining with the following monoclonal antibodies: negative gating for staining with LIVE/DEAD Aqua (Invitrogen; #L34957), CD14 (BD Biosciences; #564444), CD19 (BioLegend; #302242), CD56 (BD Biosciences; #564058), and CD4 (Invitrogen; #Q10008) to exclude all dead cells and monocytes, B cells, natural killer cells, and CD4 T cells, followed by positive gating on CD3 (BD Biosciences; #557943) and CD8 (Invitrogen; #MHCD0817) to selectively sort 15,000 cells from a pure CD8 population. Purity tests for sorting were performed routinely. RNA was purified from CD8 T cells using RNeasy Micro Kits (Qiagen). Libraries of T cell RNA samples were prepared using Illumina's Nextera XT DNA Library Prep Kit and sequenced on Illumina NovaSeq 6000. The libraries were generated following the Library Prep Kit manual. The libraries were pooled and prepared for sequencing following the “Standard Normalization Method” within the Illumina Novaseq6000 System Denature and Dilute Libraries Guide. The BCL files were demultiplexed using Illumina's bcl2fastq v2.20.0.422. Reads in FASTQ files were aligned to GRCh38 human reference genome and transcriptome with Hisat2. Tags per million reads were calculated from aligned reads using StringTie.

Dimensionality reduction and clustering

t-SNE dimensionality reduction of cells based on whole transcriptomes was generated by 10× Genomics Cell Ranger pipeline (version 3.0), as reported previously. Larkin et al., N. Engl. J. Med. 381:1535-1546 (2019). Dimensionality of gene-barcode matrices was first reduced to 10 principal components using principal-component analysis (PCA). PCA-reduced data were further reduced to 2D space using the t-SNE method and visualized in the Loupe Cell Browser (10× Genomics) and/or R. Graph-based clustering of cells was conducted in the PCA space; a sparse nearest-neighbor graph of the cells was built first, and Louvain modularity optimization was then applied. The number of nearest neighbors was logarithmically in accordance with the number of cells. In the last step, repeated cycles of hierarchical clustering and merging of cluster pairs that had no significant differential expression were performed until no more cluster pairs could merge.

3D t-SNEs of cells were calculated using the “cellranger reanalyze” command with modified parameters and visualized using R plotly package.

### Gene Differential Expression of Clusters

Gene differential expression analyses of each cluster were conducted by cellranger (10× Genomics). The log2 fold change of a certain gene's expression (UMIs) in one cluster compared with all other clusters, and the corresponding adjusted P values, were calculated for each cluster.

### Transcriptional Similarity of Clusters

To compare similarity between the three shared clusters (2, 6, and 15) and other clusters, Pearson correlations were calculated between each pair using the clusters' average gene expressions (mean UMIs of genes). The results were visualized using Circos Table Viewer, with ribbons connecting two clusters representing significant similarity (50th percentile of all Pearson distances) between them.

### PD-1 Coexpression Analyses

Coexpression patterns of genes with PD-1 (PDCD1) were calculated in T cells of the three shared clusters (clusters 2, 6, and 15). Pearson correlations of expression levels were calculated between PD-1 and 13,411 genes that were expressed (UMI>1) in more than 0.1%

$$\frac{\begin{matrix}{\sum }_{i = 1}^{n} & ( {p}_{i}\end{matrix}}{\sqrt{\begin{matrix}{\sum }_{i = 1}^{n} & {\overline{p} )}^{2}\end{matrix}} \sqrt{{\sum }_{i = 1}^{n}}} ,$$

of the total cells using R and the following formula:
where n is total number of cells in the three shared clusters; pi is expression (UMI) of PD-1 in the cell i; gi is expression (UMI) of the query gene in cell i;p⁻ and g⁻ are mean expression of PD-1 and the query gene in the total n cells, respectively; and rpg is the Pearson correlation coefficient between PD-1 and the query gene.

The t statistics of Pearson correlation were calculated by the following formula:

$$\frac{{r}_{p g} \sqrt{\begin{matrix}n & 2\end{matrix}}}{\sqrt{\begin{matrix}1 & {r}_{pg}^{2}\end{matrix}}} ,$$

where rpg is correlation coefficient of PD-1 and the query gene and n is the number of samples (cells).

P values of Pearson correlation were probabilities of T>t or <−t, where T follows at distribution with n−2 degrees of freedom.

The P values were adjusted with FDR correction, and the genes of significant adjusted P values (<0.05) were then ranked by their Pearson correlation coefficient r calculated in the three shared clusters from the most significant to the least significant correlation.

### Cell Signaling Enrichment Analyses

Signaling pathway enrichment and upstream regulator prediction were analyzed using Ingenuity Pathway Analysis (IPA; Qiagen), unless indicated specifically.

### Cell Trajectory Analysis

Single-cell trajectories of cells were built using the Monocle package (version 2.8.0) as previously reported. Larkin et al., N. Engl. J. Med. 381:1535-1546 (2019). Briefly, whole-transcriptome trajectories of T cells were built using 9,080 genes that were expressed (UMI >1) in more than 1% of the total T cells. Dimensionality reduction was conducted using the DDRTree method, and the minimum spanning tree was plotted using the plot_cell_trajectory function.

Expression levels of the signature genes along trajectories (i.e., from the cluster 2 end to the cluster 6 or cluster 15 end) were visualized using the plot_genes_branched_heatmap function; on the generated heatmap, gene expression levels were smoothened using the VGAM package, rescaled to a −3 to 3 range, and hierarchically clustered.

### Prognosis Model Building and Validating

The prognosis model to predict response of patients to ICI therapy was built using PD-1 and the top 20 genes of the PD-1 coexpression genes (21 predictor genes in total) that were mostly correlated (most significant P values) with PD-1. Responders/non-responders were defined following the RECIST criteria (i.e., complete response and partial response for responders or stable disease and progressive disease for non-responders. Eisenhauer et al., Eur. J. Cancer. 45:228-247 (2009).

The Gene Expression Omnibus (GEO) database was surveyed for all available data that were provided with both T cell transcriptomics and matching patient treatment response records to immune checkpoint therapy. A total of two published datasets, GSE120575 and GSE123813, were returned from this survey, and both were used for this study. The inventors randomly assigned GSE120575 as training data, and GSE123813, along with their two additional datasets, were used as independent validation sets.

Model training and testing were conducted using R. The training data (GSE120575) provided scRNaseq of immune cells collected before and after immune from melanoma patients treated with checkpoint inhibitors. We used all 1,802 tumor-infiltrating CD8 T cells (as identified in the article of that dataset) that were collected before ICI therapy for model training. The 21 predictor genes identified by the inventors' co-expression analyses were used as independent variables, and the response to ICI was applied as the dependent variable (1, responder; 0, non-responder). Logistic regression was performed using these independent variables and dependent variables from the dataset based on the following equation to calculate β0 and βi for the model:

$$P ( Y = 1 ) = \frac{1}{1 + {e}^{- ( {β}_{0} + {\sum }_{i = 1}^{k} {β}_{i} {X}_{i} )}} ,$$

where Y is the dependent variable, Xi is the independent variable, k is the number of independent variables, P is probability of a 1 value of Y, and β0 and βi are model parameters to be determined in the regression.

The model with β0 and βi determined from the regression test were applied to calculate P (probability) for each individual CD8 T cells from the dataset and returned with a predicted status (1, responder; 0, non-responder). Subsequently, the proportions of all calculated CD8 T cells in one sample were calculated and presented for each patient with the scored output using the abovementioned formula.

The model was then further validated in two additional datasets. Dataset 1 was from a previously published study (GSE123813), in which CD8 tumor-infiltrating T cells were collected before ICI therapy from three and six patients who responded or did not respond to anti-PD-1 therapy, respectively. The response status of each T cell was predicted, and the percentage of predicted responding/nonresponding T cells in each patient was calculated. NRSs of patients were calculated by scaling the percentage of predicted nonresponding T cells to a 0˜10 range. Patients that had NRSs higher than 5.0 (more than 50% of T cells are predicted to respond) were considered responders to ICI therapy; otherwise, patients were predicted as non-responders. Dataset 2 was collected in the present study, including peripheral CD8 T cells of 32 melanoma patients (15 responders and 17 non-responders) profiled by RNaseq. Patients' gene expression levels were input into out prediction model. For CD8 T cell scRNaseq data, the model predicted status of each T cells, and the ratio of nonresponding T cells in a patient was rescaled to 0˜10 as the patient's NRS. For bulk RNaseq data, the output values of the inventors' logistic regression model (ranging 0˜1) were rescaled to 0˜10 as patients' NRSs. Patients that had NRSs higher than 5.0 were considered responders to ICI therapy. The P values comparing the NRSs between true responders and true non-responders were calculated by t test. A P value <0.05 is considered significant.

### Statistics

Unless otherwise stated, P values of gene differential expression were determined by two-tailed Welch's t test. P values of enrichment of pathways, upstream regulators, and gene ontology terms were generated by the corresponding bioinformatics tools. All statistics calculations were conducted using R unless otherwise stated.

To determine the size of samples for validating our NiCir model, power calculation were conducted to determine the required sample size for NiCir using the rocr package in R. Power calculation suggested that in a balanced feature model (kappa=1), with a power of 0.9 and a significance level of 0.05 for the logistic regression prediction model, a proper sample size should be at least 14 samples (seven responders and seven non-responders), as shown in this case of NiCir for a AUC of 0.9 (as indicated in the inventors' training set, FIG. 8B), a proper sample size should be at least 14 samples (seven responders and seven non-responders). The inventors validated the NiCir model using nine previously published samples (Yost et al., Nat. Med. 25:1251-1259 (2019)) and 32 samples collected in the present study (FIG. 8C). They collected samples from both responders and non-responders to ensure a proper kappa=1 for the model evaluation.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating cancer in a subject in need thereof, comprising
determining the level of expression of a signature gene in a lymphocyte sample from the subject; and selecting the type of cancer treatment for the subject based on the differential level of expression of one or more signature genes.

2. The method of claim 1, wherein the signature genes are selected from the group consisting of TUBB, TUBA1B, HIST1H4C, HMGB2, H2AFZ, FABP5, HMGN2, HMGB1, COTL1, TPI1, CALM3, ACTB, PSMB9, CALM2, CLIC1, CD74, CST7, LSP1, SRGN, HLA-C, NKG7, LAG3, PDCD1, HLA-A, STMN1, GAPDH, PD1, and RPL13A.

3. The method of claim 1, wherein the cancer is melanoma.

4. The method of claim 1, further comprising the step of obtaining a lymphocyte sample from the subject.

5. The method of claim 1, wherein the lymphocyte sample comprises a blood sample.

6. The method of claim 1, wherein the lymphocytes comprise peripheral blood lymphocytes.

7. The method of claim 1, wherein the lymphocytes comprise CD8+ TOXPHOS cells.

8. The method of claim 1, wherein the expression level of a plurality of signature genes is evaluated.

9. The method of claim 1, wherein the level of gene expression is determined using flow cytometry incorporating RNA hybridization.

10. The method of claim 1, wherein cancer treatment comprises cancer immunotherapy.

11. The method of claim 10, wherein the cancer immunotherapy comprises administration of anti-PD1, anti-PDL1, or anti-CTL4 antibodies.

12. the method of claim 1, wherein the cancer treatment comprises immune checkpoint inhibitor therapy.

13. The method of claim 1, wherein the subject has been receiving a first type of cancer treatment, the expression of one or more signature genes is increased, and selecting the type of cancer treatment comprises selecting a different, second type of cancer treatment.

14. The method of claim 13, wherein the first type of cancer treatment comprises immunotherapy, and the second type of cancer treatment comprises a method of cancer treatment other than immunotherapy.
